SEQUENCE LISTING (5 sequences)

SEQ ID NO: 1 - protein, 1331 aa, human
MRGAPARLLLPLLPWLLLLLAPEARGAPGCPLSIRSCKCSGERPKGLSGGVPGPARRRVVCSGGDLPEPPEPGLLPNGTVTLLLSNNKITGLRNGSFLGLSLLEKLDLRNNIISTVQPGAFLGLGELKRLDLSNNRIGCLTSETFQGLPRLLRLNISGNIFSSLQPGVFDELPALKVVDLGTEFLTCDCHLRWLLPWAQNRSLQLSEHTLCAYPSALHAQALGSLQEAQLCCEGALELHTHHLIPSLRQVVFQGDRLPFQCSASYLGNDTRIRWYHNRAPVEGDEQAGILLAESLIHDCTFITSELTLSHIGVWASGEWECTVSMAQGNASKKVEIVVLETSASYCPAERVANNRGDFRWPRTLAGITAYQSCLQYPFTSVPLGGGAPGTRASRRCDRAGRWEPGDYSHCLYTNDITRVLYTFVLMPINASNALTLAHQLRVYTAEAASFSDMMDVVYVAQMIQKFLGYVDQIKELVEVMVDMASNLMLVDEHLLWLAQREDKACSRIVGALERIGGAALSPHAQHISVNARNVALEAYLIKPHSYVGLTCTAFQRREGGVPGTRPGSPGQNPPPEPEPPADQQLRFRCTTGRPNVSLSSFHIKNSVALASIQLPPSLFSSLPAALAPPVPPDCTLQLLVFRNGRLFHSHSNTSRPGAAGPGKRRGVATPVIFAGTSGCGVGNLTEPVAVSLRHWAEGAEPVAAWWSQEGPGEAGGWTSEGCQLRSSQPNVSALHCQHLGNVAVLMELSAFPREVGGAGAGLHPVVYPCTALLLLCLFATIITYILNHSSIRVSRKGWHMLLNLCFHIAMTSAVFAGGITLTNYQMVCQAVGITLHYSSLSTLLWMGVKARVLHKELTWRAPPPQEGDPALPTPSPMLRFYLIAGGIPLIICGITAAVNIHNYRDHSPYCWLVWRPSLGAFYIPVALILLITWIYFLCAGLRLRGPLAQNPKAGNSRASLEAGEELRGSTRLRGSGPLLSDSGSLLATGSARVGTPGPPEDGDSLYSPGVQLGALVTTHFLYLAMWACGALAVSQRWLPRVVCSCLYGVAASALGLFVFTHHCARRRDVRASWRACCPPASPAAPHAPPRALPAAAEDGSPVFGEGPPSLKSSPSGSSGHPLALGPCKLTNLQLAQSQVCEAGAAAGGEGEPEPAGTRGNLAHRHPNNVHHGRRAHKSRAKGHRAGEACGKNRLKALRGGAAGALELLSSESGSLHNSPTDSYLGSSRNSPGAGLQLEGEPMLTPSEGSDTSAAPLSEAGRAGQRRSASRDSLKGGGALEKESHRRSYPLNAASLNGAPKGGKYDDVTLMGAEVASGGCMKTGLWKSETTV

SEQ ID NO: 2 - DNA, 25 bp, Artificial Sequence
  oligonucleotide
actggagaggtcactgcgcagatta

SEQ ID NO: 3 - DNA, 25 bp, Artificial Sequence
  oligonucleotide
ggatgcggacgcagggtcccgacat

SEQ ID NO: 4 - DNA, 25 bp, Artificial Sequence
  oligonucleotide
actcgacaggtgactgcacagatta

SEQ ID NO: 5 - DNA, 620 bp, zebrafish
cgcggatccgaacactgcgtttgctggctttgatgaaaactcacagtttggaaaggagtaactttttaacaattacagtttctataagctcggatactttaagcagttatcactcaaaagtttatgccctgatgtcatgggctgacctgggaccgcatagttgatagtgtgggggacaatgtcgggaccctgcgtccgcatcccgttttgggttcgcgtgtttctcctgctgctgctttatagaatcgctgcgggctgcccggagcttttttccagcggctgcagctgcacggaggaccgcagtaaagcccaccctactcccgggactcggaggaaagtgagctgcggtggaaaggagctgactgaaacgcccgaagtcagtctgctccccaacaggactgtctctctaaatctgagcaacaaccgtattcgcatgctgaaaaatggctccttcgctggcttatcctcccttgagaagctggatctcaggaataacttgatcagcaccattatgcccggagcctttctgggtctcacagcacttcgaaaacttgacctctccagtaaccgaattgggtgcttgactccagaaatgttccagggacttaccaacctcacta

(19) United States
(12) Patent Application Publication
(10) Pub. No.: US 2010/0278810 A1
(43) Pub. Date: Nov. 4, 2010

(54) Methods of Modulating Angiogenesis

(76) Inventors: Frank Kuhnert, Palo Alto, CA (US); Hsiao-Ting Wang, Redwood City, CA (US); Calvin Jay Kuo, Palo Alto, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/600,984

(22) PCT Filed: May 23, 2008
(86) PCT No.: PCT/US08/06594

§ 371 Date: Jun. 16, 2010

Related U.S. Application Data
(60) Provisional application No. 60/940,026, filed on May 24, 2007.

(51) Int. Cl. A61K 39/395; A61K 31/711; A61K 31/7105; A61K 31/713; A61P 25/00; A61P 9/10; A61P 3/10; A61P 35/00

(52) U.S. Cl. 424/130.1; 514/44 A; 514/44 R

(57) ABSTRACT

The present invention provides methods of modulating angiogenesis in an individual, the methods generally involving administering to an individual an agent that modulates the expression or activity of GPR124. In one embodiment, the methods of the invention relate to inhibiting pathological angiogenesis by decreasing activity of GPR124, which method may be carried out in conjunction with administration of one or more other anti-angiogenic agents.

### BACKGROUND OF THE INVENTION

Angiogenesis and vasculogenesis are processes involved in the growth of blood vessels. Angiogenesis is the process by which new blood vessels are formed from extant capillaries, while vasculogenesis involves the growth of vessels deriving from endothelial progenitor cells. The development of the vascular system, one of the earliest events in organogenesis, begins with vasculogenesis, during which angioblasts differentiate into endothelial cells and assemble into a primitive vascular plexus. Following vasculogenesis is the growth, expansion and remodeling of primitive vessels into a mature vascular network, a process known as angiogenesis. Angiogenesis plays an important role in both physiological as well as pathological situations. During embryonic development, angiogenesis is critical in providing growing organs with the necessary oxygen to develop. Later, in the adult setting, angiogenesis occurs during ovulation, placental development and wound healing. Many events that occur during normal vascular development in the embryo are recapitulated during adult angiogenesis. Because angiogenesis is the result of a delicate balance between pro- and anti-angiogenic factors, disruption of this balance results in inappropriate vessel growth.

Angiogenesis is a complex, combinatorial process that is regulated by a balance between pro- and anti-angiogenic molecules. Angiogenic stimuli (e.g. hypoxia or inflammatory cytokines) result in the induced expression and release of angiogenic growth factors such as vascular endothelial growth factor (VEGF) or fibroblast growth factor (FGF). These growth factors stimulate endothelial cells (EC) in the existing vasculature to proliferate and migrate through the tissue to form new endothelialized channels.

Angiogenesis and vasculogenesis also contribute to pathologic conditions such as tumor growth, diabetic retinopathy, rheumatoid arthritis, and chronic inflammatory diseases (see, e.g., U.S. Pat. No. 5,318,957; Yancopoulos et al. (1998) Cell 93:661-4; Folkman et al. (1996) Cell 87; 1153-5; and Hanahan et al. (1996) Cell 86:353-64).

Both angiogenesis and vasculogenesis involve the proliferation of endothelial cells. Endothelial cells line the walls of blood vessels; capillaries are comprised almost entirely of endothelial cells. The angiogenic process involves not only increased endothelial cell proliferation, but also comprises a cascade of additional events, including protease secretion by endothelial cells, degradation of the basement membrane, migration through the surrounding matrix, proliferation, alignment, differentiation into tube-like structures, and synthesis of a new basement membrane. Vasculogenesis involves recruitment and differentiation of mesenchymal cells into angioblasts, which then differentiate into endothelial cells which then form de novo vessels (see, e.g., Folkman et al. (1996) Cell 87:1153-5).

Inappropriate, or pathological, angiogenesis is involved in the growth of atherosclerotic plaque, diabetic retinopathy, degenerative maculopathy, retrolental fibroplasia, idiopathic pulmonary fibrosis, acute adult respiratory distress syndrome, and asthma. Furthermore, tumor progression is associated with neovascularization, which provides a mechanism by which nutrients are delivered to the progressively growing tumor tissue.

While the concept of slowing or even halting the progression of cancer by targeting its blood supply was first proposed more than 30 years ago (Folkman, 1971), angiogenesis inhibitors are only now entering the mainstream of cancer therapeutics (Hurwitz et al., 2004). The success of Avastin, a monoclonal antibody raised against Vascular Endothelial Growth Factor (VEGF), in treating colon cancer brings hope for the use of angiogenesis inhibitors for the treatment of other malignancies such as prostate cancer—one of the most common cancers in men (Young, 2002). There is a need in the art for methods of reducing pathological angiogenesis. The present invention addresses this need.

Publications. U.S. Pat. No. 6,733,990, Hodge et al. May 11, 2004 “Nucleic acid encoding 15571, a GPCR-like molecule of the secretin-like family”. U.S. Pat. No. 6,570,003 Hu et al. May 27, 2003, “Human 7™ proteins and polynucleotides encoding the same”. Carson-Walter et al. (2001) Cancer Research 61:6649-6655, “Cell surface tumor endothelial markers are conserved in mice and humans”. St Croix et al., Science, 2000, 289, 1197-1202. United States Patent Application 20040023378, Chiang, Ming-Yi et al. Feb. 5, 2004; “Antisense modulation of KIAA1531 protein expression”.

### SUMMARY OF THE INVENTION

The present invention, provides methods of modulating angiogenesis in an individual. It is demonstrated herein for the first time that GPR124 is functionally required for angiogenesis in mammals; and is involved in a VEGF independent signaling pathway. In particular, GPR124 is expressed during development of the vasculature. In addition, GP124 acts to regulate expression of molecules involved in the blood brain barrier, e.g. glut1 transporter. In adults, GPR124 is almost exclusively expressed on endothelial cells in the central nervous system; whereas outside of the CNS it is largely expressed in pericytes. Upregulation of GPR124 acts to increase expression of barrier proteins.

The methods of the invention generally involve administering to the individual an effective amount of a GPR124 modulating agent. The methods are useful to treat conditions associated with, or resulting from, angiogenesis, including pathological angiogenesis. Inhibition of GPR124 also acts to decrease BBB, e.g. by down-regulating glut1 expression. The invention further provides methods of treating a condition associated with or resulting from neovascularization, or angiogenesis. In other embodiments, methods are provided for enhancing angiogenesis.

The present invention includes a method of reducing angiogenesis in a mammal. The method generally involves administering to a mammal a GPR124 antagonist in an amount effective to reduce angiogenesis. The present invention also features method of treating a disorder associated with pathological angiogenesis. In some embodiments, the invention features a method of inhibiting a proliferative retinopathy in a mammal. The methods generally involve administering to a mammal a GPR124 antagonist in an amount effective to reduce pathological angiogenesis. In some embodiments, the methods further comprise administering a second angiogenesis inhibitor. GPR124 is demonstrated herein to operate in a pathway independent from VEGF, and thus providing for additive or synergistic effects in combination with VEGF inhibition, as relevant to anti-angiogenic therapy of cancer and ocular disorders.

The present invention further features a method of inhibiting tumor growth in a mammal. In some embodiments, the invention features a method of inhibiting pathological neovascularization associated with a tumor. The methods generally involve administering to a mammal a GPR124 antagonist in an amount effective to reduce angiogenesis associated with a tumor. In some embodiments, the invention further comprises administering an anti-tumor chemotherapeutic agent other than a GPR124 antagonist.

GPR124 is selectively involved in central nervous system angiogenesis, with highly restricted expression of GPR124 in CNS endothelial cells, including brain and retina. This strong tropism for the CNS—both functionally and in expression—stands in marked contrast to endothelial receptor systems such as VEGFNEGFR, Angiopoietin/Tie2, EphinB2/EphB4 and Notch/DLL4. In some embodiments of the invention, angiogenic activity in the CNS is selectively inhibited by downregulating the activity of GPR124, for example during CNS tumorigenesis, macular degeneration, diabetic retinopathy, and the like. In other embodiments, angiogenesis of the CNS is enhanced by increasing GPR124 activity, e.g. in treatment of ischemic stroke, and the like.

The retina is contiguous with and is an extension of the central nervous system. GPR124 expression is found in all retinal microvasculature, in both endothelial and pericyte compartments. In some embodiments of the invention, GPR124 inhibition is utilized in the treatment of over-vascularization that is pathogenic for macular degeneration and diabetic retinopathy.

In tissues outside of the CNS and liver, GPR124 expression is largely confined to pericytes. In adult organs GPR124 expression is exclusively vascular, but in pericytes, not endothelial cells. In some embodiments of the invention, angiogenesis is modulated in pericytes by altering expression and/or activity of GPR124. For example, pericyte activity in angiogenesis may be inhibited by decreasing GPR124 expression or activity. Pericytes may also be used in screening assays for identifying and developing agents that alter GPR124 mediated angiogenesis, e.g. by inhibiting pericyte migration or proliferation in culture.

### BRIEF DESCRIPTION OF THE DRAWINGS

FIG. 1. GPR124 is predicted to encode a seven-pass transmembrane protein characteristic of members of the G protein coupled receptor (GPCR) family. The amino terminal extracellular region, which is approximately 760 amino acids long, contains four simple leucine rich repeats (LRR), one carboxy-terminal type LRR, one immunoglobulin-type domain and one putative hormone-receptor domain followed by a GPCR proteolysis site (GPS).

FIG. 2. A clone encoding the murine GPR124 genomic locus was isolated from a 129sV BAC library. From this clone, a targeting construct was produced containing a 4.0 kb 5′ homology arm and a 2.7 kb 3′ arm, and in which a SacII fragment of exon 1 (including the start codon) was replaced with a lacZ reporter (SDKlacZpA) and a neomycin selection cassette (PGKneopA). This construct was linearized and electroporated into mouse ES cells.

FIG. 3. Disruption of the GPR124 locus in ES cells and GPR124^{-/-} embryos. A. Southern blot. Genomic DNA was prepared from G418-resistant ES clones previously electroporated with the targeting construct described in FIG. 2. Southern blotting performed with the 5′ probe depicted in FIG. 2 revealed homologous recombination and correct targeting in two clones, with a characteristic 4.5 kb EcoRV fragment. B. Northern blot. Total RNA from E12.5 GPR124^{+/-} and GPR124^{-/-} embryos was analyzed by Northern blot revealing absence of transcript in homozygous mutant embryos. C. Real-time PCR: Total RNA from E12.5 GPR124^{+/-} and GPR124^{-/-} embryos was analyzed by real-time PCR revealing >3-log reduction in GPR124RNA in ko animals.

FIG. 4. CNS hemorrhage in GPR124^{-/-} animals. [a,b]. Gross appearance of embryos. [c]. H&E section of E12.5 forebrain demonstrating hemorrhage in the neuroepithelium and ventricle. *-forebrain hemorrhage. Nt=neural tube hemorrhage. L=liver, which appears red because of physiologic erythropoiesis. Hemorrhage is confined to the CNS.

FIG. 5. Transverse frozen sections from wild-type E14.5 embryos were analyzed for by immunofluorescence for GPR124 (left panel) or CD31 (middle panel) expression. A merged image is shown in the right panel, demonstrating complete merge of the signal, consistent with brain endothelial expression.

FIG. 6. The vascular patterning of GPR124 wild-type (+/+, left panel) versus knockout (−/−, right panel) embryonic E12.5 brain was analyzed by laminin immunofluorescence. Angiogenesis in wild-type telencephalon (left panel) occurs efficiently with efficient migration of endothelial cells to the ventricular border. In contrast, endothelial migration to the periventricular area is severely impaired in GPR124 knockout embryos, consistent with a severe defect in brain angiogenesis (right panel) This is illustrated by the boxed areas which are replete with endothelial cells wild-type but devoid of endothelium in the GPR124 knockout. V=ventricle.

FIG. 7. Brain angiogenesis in GPR124 wild-type (+/+, left panel) versus knockout (−/−, right panel) embryonic E14.5 brain was analyzed by laminin immunofluorescence. Angiogenesis in wild-type telencephalon (left panel) occurs efficient migration of endothelial cells to the ventricular border. In contrast, endothelial migration to the periventricular area is severely impaired in GPR124 knockout embryos, consistent with a severe defect in brain angiogenesis (right panel) This is illustrated by the boxed areas which are replete with endothelial cells wild-type but devoid of endothelium in the GPR124 knockout. Note the presence of large glomeruloid vascular malformations in knockout but not wild-type (arrows). V=ventricle.

FIG. 8. Brain angiogenesis in GPR124 wild-type (+/+, left panel) versus knockout (−/−, right panel) embryonic E15.5 brain was analyzed by laminin immunofluorescence. Angiogenesis in wild-type telencephalon (left panel) occurs efficiently with efficient migration of endothelial cells to the ventricular border. In contrast, endothelial migration to the periventricular area is severely impaired in GPR124 knockout embryos, consistent with a severe defect in brain angiogenesis (right panel) This is illustrated by the boxed areas which are replete with endothelial cells wild-type but devoid of endothelium in the GPR124 knockout. Note the presence of large glomeruloid vascular malformations in knockout but not wild-type (arrows). V=ventricle.

FIG. 9. Ultrastructure of vascular malformations in E12.5 GPR124 knockout brain (telencephalon). Top panels: Confocal microscopy demonstrates the presence of giant glomeruloid vascular aggregates in knockout (right, arrows) but not wild-type (left, arrows) brain. The extra-cranial vasculature of the perivenous plexus (pvp) is unaltered in both. Red-CD31. Green-PDGFRβ. Bottom panels: Electron microscopy reveals a haphazard organization of superfluous endothelial cells in knockout (right,) but not wild-type (left) brain.

FIG. 10. Angiogenesis of the developing neural tube (spinal cord) is defective in GPR124 knockout embryos. Vascular invasion of the neural tube is impaired in GPR124 knockout (−/−, right panels) versus GPR124 wild-type (+/+, left panels). The angiogenic deficit is most pronounced in the ventral neural tube (yellow enclosed region) where little CD31 signal (red, endothelial marker) is observed in knockout as opposed to abundant signal in wild-type. Red-CD31. Green-nestin.

FIG. 11. Neural tube angiogenesis in GPR124 knockout (−/−, top panel) versus wild-type (+/+, bottom panel) embryonic E14.5 brain was analyzed by laminin immunofluorescence. Note the presence of large glomeruloid vascular malformations in knockout but not wild-type (arrows).

FIG. 12. GPR124 gene deletion does not affect angiogenesis of non-CNS organs. GPR124 knockout (−/−, right panels) or wild-type (+/+, left panels) embryos were harvested at E14.5 and frozen sections of the indicated organs analyzed by CD31 (endothelial marker) immunofluorescence. This revealed that angiogenesis in non-CNS vascular beds was unaltered by GPR124 gene deletion, in contrast to severe effects in brain and neural tube.

FIG. 13. GPR124 is expressed in a pan-vascular fashion in adult brain. Frozen sections of C57Bl/6 adult mouse brain were examined for expression of GPR124 and CD31 by immunofluorescence. In both cerebrum (left panels) and cerebellum (right panels), strong co-localization is observed between GPR124 (green) and CD31 (endothelial marker, red), yielding yellow/orange signal indicative of endothelial GPR124 expression which extends over virtually every capillary bed.

FIG. 14. GPR124 is expressed in both endothelial cells and pericytes of the adult brain. Frozen sections of C57Bl/6 adult mouse brain were examined for expression of GPR124, CD31 and PDGFRβ by immunofluorescence. In panel B., co-localization is observed between GPR124 (red) and CD31 (endothelial marker, green), yielding yellow signal indicative of endothelial GPR124 expression (arrow). Pericyte expression of GPR124 is indicated by diffuse punctate red signal over the pericyte body (*). Additionally, in panel C., co-localization is observed between GPR124 (red) and PDGFRβ (pericyte marker, green), again evidenced by punctate red signal over the pericyte body (*). Expression of GPR124 alone in brain vasculature is depicted in panel D.

FIG. 15. GPR124 is expressed on brain endothelial cells. (A). FACS Analysis of GPR124 expression in primary wild-type E12.5 brain endothelial cells. Note that all CD31-positive endothelial cells also express GPR124. (B). In contrast, FACS of CD31-positive endothelial cells from knock-out embryos reveals complete lack of GPR124 expression; compare upper right quadrants in B (ko) versus A (wildtype). (C,D). BEnd3 brain endothelial cells show robust expression of both GPR124 (C, blue trace) and CD31 (D, blue trace) as analyzed by FACS. Secondary antibody controls are shown in red.

FIG. 16. Inhibition of tumor growth and angiogenesis by systemic adenoviral delivery of GPR124 ectodomain. C57Bl/6 mice bearing pre-established T241 fibrosarcoma (n=10) received single iv injection of 109 pfu of adenovirus expressing a GPR124 ectodomain-Fc fusion protein or a control immunoglobulin IgG2a Fc fragment. (A). Tumor growth inhibition. (B). Reduction of pericyte content by GPR124 ectodomain-Fc fusion adenovirus as opposed to the Fc control as indicated by reduced NG2 staining (pericyte marker, green) and free CD31-expressing cells (endothelial marker, red) (arrows) which are not associated with green NG2-positive cells. This is consistent with primary expression of GPR124 in pericytes, not endothelial cells in T241 fibrosarcoma.

FIG. 17. Cloning of full length zebrafish gpr124. The previously undeposited 5′ end of the zebrafish gpr124 homolog was cloned by 5′ RACE (rapid amplification of cDNA ends), allowing subsequent cloning of full length zebrafish gpr124. 5′RACE extended the zebrafish sequence by 193 aa. The extended zebrafish gpr124 ORF encodes a protein of 1367 aa which is 49% identical to murine gpr124 and 54% identical to human gpr124. The start codon is contained within the newly extended sequence and is underlined. Like its murine and human homologs, zebrafish gpr124 encodes a 7-pass transmembrane protein with a large N-terminal extracellular region which is comprised of five LRRs, one immunoglobulin-type domain (IgG), one putative hormone-receptor domain (HR) and one GPCR proteolysis site (GPS) The newly extended 5′ region is designated by a box.

FIG. 18. Vascular expression of zgpr124 at 3 and 4 dpf. Expression is noted in the cerebral vasculature at this stage, in the middle cerebral vein, as well as the primordial midbrain channel and aortic arches (left panels). In all of the developmental stages examined, gpr124 expression (left panels) closely resembles the expression pattern of VE-cadherin (right panels), which is specifically expressed in the vascular endothelial cells in both developing tissue and mature vasculature.

FIG. 19. Pericardial edema (arrow), characteristic of vascular insufficiency, is observed in zebrafish embryos injected with the ½ splice MO (bottom) but not the 5mis control MO (top).

FIG. 20. At 3 dpf, MO injected embryos began to exhibit angiogenic deficits in the head. Whole-mount fluorescence imaging of ½ MO-injected Flk1-GFP embryos exhibited grossly abnormal aortic arch arteries, with a pronounced lack of anterior extension of AA1 as well as lack of lateral extension of the more posterior arch arteries. These were not seen with 5mis, a negative control morpholino with a 5 by mismatch that does not cause a phenotype. 1/2 MO indicates the splice retention morpholino targeting the 1/2 splice junction of zGpr124. The yellow border indicates abnormally avascular areas induced by the 1/2 MO. The absence of rostral extension of head vasculature is indicated (*).

FIG. 21. GPR124 regulates expression of the Blood-Brain Barrier (BBB) transporter Glut1. E14.5 embryo forebrain was analyzed for endothelial cells (isolectin B4=IB4) cells and Glut1. Note colocalization of IB4 and Glut1 in w.t. (A, B) but not GPR124 ko (C, D). The BBB transporter Glut1 is down-regulated in GPR124 vasculature indicating that GPR124 regulates expression of the Blood-Brain Barrier transporter Glut1. Arrows denote glomeruloid malformations. v=ventricle.

FIG. 22. Ectopic expression of GPR124 in liver vasculature results in induction of barrier function in liver sinusoidal endothelium. Biotin was injected i.v. into either w.t. (left panel) or transgenic Tie2-GPR124 mice (right panel) followed by harvest of the liver and analysis of biotin extravasation by strepavidin-FITC staining of frozen liver sections. Note that the w.t. hepatic vasculature is inherently leaky as indicated by tracer leakage resulting in obscurement of the sinusoidal vascular pattern (left panel). However, in transgenic Tie2-GPR124 mice overexpressing in the liver vasculature, barrier properties are induced, resulting in markedly enhanced retention of the tracer within the sinusoids, and allowing the sinusoids to be clearly visualized (right panel).

FIG. 23. Overexpression of GPR124 produces CNS vascular hyperplasia. Histologic examination of transgenic Tie2-GPR124 mice overexpressing GPR124 in both CNS and non-CNS endothelium was performed. This revealed vascular hyperplasia in the CNS of transgenic (right panels) but not wild-type (left panels) mice, indicating that GPR124 functions as a pro-angiogenic receptor and that GPR124 stimulation could be used to induce CNS angiogenesis.

FIG. 24. Phenocopy of the GPR124 ko phenotype by endothelial GPR124 deletion. Conditional GPR124 ko mice were crossed to Tie2-Cre mice expressing Cre in endothelium. Forebrain hemorrhage (arrow) is seen in GPR124flox/-GPR124flox/−Tie2-Cre(+) embryos (panel B) but not in unexcised littermate controls without Cre expression (GPR124flox/-Tie2-Cre(−)) (panel A). CD31 IF demonstrates forebrain glomeruloid malformations and impaired angiogenic migration in excised GPR124flox/-Tie2-Cre(+) embryos (panel D, magnified image in E), but not in unexcised GPR124flox/-Tie2-Cre(−) controls (panel C). E14.5 embryos. v=ventricle.

### DETAILED DESCRIPTION OF THE EMBODIMENTS

GPR124, also known as tumor endothelial marker 5 (TEM5), encodes a seven-pass transmembrane protein (1329 amino acids), characteristic of members of the G protein coupled receptor (GPCR) family. The genetic sequence of the human GPR124 (NCBI GenelD:25960) is publicly available at in the chromosome 8 sequence; NC_{—}000008.9 (37773931.37820649), and having the amino acid sequence:

| | |
|---|---|
| 1 | mrgaparlll pllpwlllll apeargapgc plsirsckcs gerpkglsgg vpgparrrvv |
| 61 | csggdlpepp epgllpngtv tlllsnnkit glrngsflgl sllekldlrn niistvqpga |
| 121 | flglgelkrl dlsnnrigcl tsetfqglpr llrlnisgni fsslqpgvfd elpalkvvdl |
| 181 | gtefltcdch lrwllpwaqn rslqlsehtl caypsalhaq algslqeaql ccegalelht |
| 241 | hhlipslrqv vfqgdrlpfq csasylgndt rirwyhnrap vegdeqagil laeslihdct |
| 301 | fitseltlsh igvwasgewe ctvsmaqgna skkveivvle tsasycpaer vannrgdfrw |
| 361 | prtlagitay qsclqypfts vplgggapgt rasrrcdrag rwepgdyshc lytnditrvl |
| 421 | ytfvlmpina snaltlahql rvytaeaasf sdmmdvvyva qmiqkflgyv dqikelvevm |
| 481 | vdmasnlmlv dehllwlaqr edkacsrivg aleriggaal sphaqhisvn arnvaleayl |
| 541 | ikphsyvglt ctafqrregg vpgtrpgspg qnpppepepp adqqlrfrct tgrpnvslss |
| 601 | fhiknsvala siqlppslfs slpaalappv ppdctlqllv frngrlfhsh sntsrpgaag |
| 661 | pgkrrgvatp vifagtsgcg vgnltepvav slrhwaegae pvaawwsqeg pgeaggwtse |
| 721 | gcqlrssqpn vsalhcqhlg nvavlmelsa fprevggaga glhpvvypct allllclfat |
| 781 | iityilnhss irvsrkgwhm llnlcfhiam tsavfaggit ltnyqmvcqa vgitlhyssl |
| 841 | stllwmgvka rvlhkeltwr apppqegdpa lptpspmlrf yliaggipli icgitaavni |
| 901 | hnyrdhspyc wlvwrpslga fyipvalill itwiyflcag lrlrgplaqn pkagnsrasl |
| 961 | eageelrgst rlrgsgplls dsgsllatgs arvgtpgppe dgdslyspgv qlgalvtthf |
| 1021 | lylamwacga laysqrwlpr vvcsclygva asalglfvft hhcarrrdvr aswraccppa |
| 1081 | spaaphappr alpaaaedgs pvfgegppsl ksspsgssgh plalgpcklt nlqlaqsqvc |
| 1141 | eagaaaggeg epepagtrgn lahrhpnnvh hgrrahksra kghrageacg knrlkalrgg |
| 1201 | aagalellss esgslhnspt dsylgssrns pgaglqlege pmltpsegsd tsaaplseag |
| 1261 | ragqrrsasr dslkgggale keshrrsypl naaslngapk ggkyddvtlm gaevasggcm |
| 1321 | ktglwksett v |

The amino terminal extracellular region, which is approximately 760 amino acids long, contains four simple leucine rich repeats (LRR), one carboxy-terminal type LRR, one immunoglobulin-type domain and one putative hormone-receptor domain followed by a GPCR proteolysis site (GPS). The hydrophobic domain of GPR124 shares homology with members of the secretin family of GPCRs (class II) while the LRR domain shares homology with LIG-1 and SLIT proteins. The mouse ortholog of human GPR124, identified by database search of mouse ESTs, shows an overall amino acid sequence identity of 88% and is most homologous at its LRR repeats and transmembrane domains, suggesting functional conservation.

Provided herein is a demonstration of the function of GPR124 through the use of multiple animal models, including a transgenic knockout mouse model in which the first coding exon has been replaced by LacZ. Whole mount lacZ staining of heterozygous GPR124^{+/LacZ} embryos reveals widespread staining in multiple vascular structures, including the myocardium of the outflow tract, dorsal aorta, carotid artery and intersomitic vessels. Homozygous knockout mice are embryonic lethal and are characterized by central nervous system hemorrhage beginning at e11.5, which is most pronounced in the embryonic forebrain and also evident in the neural tube. Immunofluorescence analysis of the brain vasculature in GPR124 knockout mice reveals large avascular periventricular areas and large glomeruloid vascular malformations near the pial surface, indicative of an angiogenic sprouting or migration defect. The vascular expression of GPR124 and the embryonic lethality and CNS vascular malformations associated with GPR124 deficiency demonstrate an essential role for GPR124 during embryonic vascular development, particularly in the CNS.

In tissues other than the CNS there is pericyte specific expression of GPR124. Capillaries are comprised of inner lining endothelial cells which are encircled by outer lining pericytes. The findings presented herein indicate that GPR124 is expressed in pericytes, not endothelial cells in the vast majority of adult organs. Further, in the tumors examined, GPR124 is expressed by tumor pericytes, not tumor endothelial cells, in marked contrast to the original description of GPR124 as a tumor endothelial marker. These data demonstrate that inhibition of GPR124 is useful for anti-angiogenic therapy targeting tumor pericytes.

GPR124 is expressed by both endothelial cells and pericytes in the brain. The notable endothelial expression of GPR124 in brain further attests to unique functions of this receptor in CNS angiogenesis. The findings presented herein indicate the utility of GPR124 inhibition for CNS tumors, e.g. in the brain, spinal cord, retina; and in the treatment of diseases involving retinal neovascularization, e.g. proliferative diabetic retinopathy and “wet” macular degeneration.

Epistasis analysis presented herein in multiple animal models indicates that GPR124 acts independently of the VEGF pathway. These data demonstrate the utility of GPR124 inhibition as an VEGF independent pathway. GPR124 inhibition can be combined with VEGF inhibition for additive or synergistic anti-angiogenic and anti-tumor effects.

The findings presented herein further indicate the utility of GPR124 activation in the stimulation of CNS angiogenesis, e.g. in treatment following ischemic stroke. The phenotype of GPR124 knockout mice indicates that endothelial cells can not fully penetrate the developing brain and spinal cord. Instead of forming a vascular network that spreads throughout the brain, endothelial cells deficient in GPR124 form large glomeruloid malformations typical of arteriovenous malformations (AVMs). AVMs are quite prevalent in the general population and are a leading cause of stroke, particularly hemorrhagic stroke. The findings presented herein indicate that GPR124 mutations could underlie AVM formation and/or stroke, where therapeutic manipulation of GPR124 can be useful in the prevention and treatment of these conditions. Analysis of single nucleotide polymorphisms in GPR124 is relevant to classification of risk and or prognosis of AVM and stroke.

The expression of GPR124 in brain endothelial cells can provide for manipulation of this receptor to modulate the blood-brain barrier (BBB). Inhibition of the BBB can be useful to increase drug delivery to the CNS, for instance for cancer. Conversely, for treatment of CNS autoimmune disorders such as multiple sclerosis, strengthening of the BBB may be desirable.

### DEFINITIONS

GPR124 is a large seven pass transmembrane protein with a large ectodomain which is classified by bioinformatic analysis as secretin-family G-protein coupled receptor. The GPR124 ectodomain consists of 5 leucine-rich repeats (LRRs), including 4 classical LRRs and one LRRCT. Additionally, the GPR124 ectodomain contains one immunoglobulin (Ig) domain, one HormR domain, and one GPS domain, the latter specifying a potential proteolytic cleavage site. During embryonic development, GPR124 is expressed in multiple vascular beds including the CNS. In adulthood, GPR124 is expressed in the endothelial cells of the central nervous system, as well as pericytes of the numerous CNS— and non-CNS organs.

Angiogenesis. Angiogenesis is a complex process characterized by two phases: the pre-vascular phase, also known as the angiogenic switch, and the vascular phase. There are five initial steps necessary for angiogenesis: 1) degradation of extracellular matrix, 2) disruption of cell adhesion, 3) increase in cell permeability, 4) proliferation of endothelial cells and 5) migration of endothelial cells toward the site of new vessel formation.

While an emphasis has been placed on endothelial cells in the angiogenic process, pericytes and vascular smooth muscle cells (vSMC), or mural cells, also play an important role. Pericytes are critical regulators of vascular morphogenesis and function. Shortly after endothelial tubes form, they become associated with mural cells. These cells provide structural support to the vessels and are important regulators of blood flow. Pericytes constitute a heterogeneous population of cells. Several functions of pericytes during angiogenesis have been proposed, including sensing the presence of angiogenic stimuli, depositing or degrading extracellular matrix and controlling endothelial cell proliferation and differentiation in a paracrine fashion. In certain diseases such as diabetic retinopathy, pericytes may be the primary affected vascular cells, which lead to secondary affects on the endothelial cells.

Studies have revealed several signaling pathways which are important during vasculogenesis and angiogenesis. One of the central mediators of both vasculogenesis and angiogenesis is vascular endothelial growth factor (VEGF). VEGF is a critical driver of vascular formation and is required to initiate formation of immature vessels and angiogenic sprouting both during embryonic development as well as in the adult. VEGF induces endothelial proliferation, promotes cell migration and inhibits apoptosis by signaling through its receptors tyrosine kinases VEGFR-1/Flt-1 and VEGFR-2/KDR/Flk-1 which are expressed specifically on the surface of vascular endothelial cells. A separate class of VEGF receptors, the Neuropilins (NRPs), has also been shown to be involved in normal vascular development as well as pathological angiogenesis.

Angiogenesis is necessary for the growth of solid tumors. Tumor vascularization is critical for the progression of a small, localized neoplasm to a large tumor with increased metastatic potential. Because tumor size is limited by oxygen and nutrient diffusion from surrounding blood vessels, the inner cells of solid tumors become necrotic when tumors exceed 2 mm in diameter unless the tumors become vascularized. As a result, angiogenesis is fundamental for the growth and metastasis of solid tumors.

Tumors provide a rich source of angiogenic factors and interactions between tumor cells, endothelial cells and stromal cells are crucial for tumor angiogenesis. Tumors secrete angiogenic factors, which stimulate endothelial cells to proliferate and secrete proteases such as matrix metalloproteinases (MMPs). MMPs are responsible for degrading the basement membrane surrounding blood vessels, thus allowing endothelial cells to migrate into surrounding tissue. These migrating endothelial cells form capillary sprouts that grow toward the tumor and eventually provide the tumor with its own blood supply.

Although many of the angiogenic pathways important for tumor vascularization are the same as those important in physiological angiogenesis, tumor vasculature is distinct. Tumor vasculature is morphologically abnormal, with tortuous, leaky vessels that have irregular diameter and thin walls. A relative deficiency of pericytes or impaired pericyte function may be partially responsible for the observed morphological features. In addition to morphological differences, tumor vessels also display molecular differences, with various cell-surface and extracellular matrix proteins specifically expressed in tumor vasculature that can be used to distinguish tumor vessels from normal vasculature.

Tumor cells secrete various cytokines and angiogenic factors that alter gene expression of endothelium growing in tumors. Molecular characterization of normal and tumor vessels by examination of differentially expressed genes can be used to identify tumor endothelial markers. In a study by St. Croix et al., gene expression profiles in endothelium derived from normal colon and colorectal cancer tissue identified 79 differentially expressed transcripts, 46 of which were elevated in tumor endothelium. The differential expression of these genes suggests that tumor and normal endothelium are different at the molecular level.

The terms “treatment”, “treating” and the like are used herein to generally mean obtaining a desired pharmacologic and/or physiologic effect, e.g., modulation of angiogenesis and/or vasculogenesis. The effect may be prophylactic in terms of completely or partially preventing a disease or symptom thereof and/or may be therapeutic in terms of a partial or complete cure for a disease and/or adverse effect. “Treatment” as used herein covers any treatment of a disease in a mammal, particularly a human, and includes: (a) preventing a disease or condition from occurring in a subject who may be predisposed to the disease but has not yet been diagnosed as having it; (b) inhibiting the disease, e.g., arresting its development; or (c) relieving the disease. In the context of the present invention, reduction of angiogenesis and/or vasculogenesis is employed for subject having a disease or condition amenable to treatment by reducing angiogenesis; and

By “therapeutically effective amount of a GPR124 antagonist” is meant an amount of a GPR124 antagonist effective to facilitate a desired therapeutic effect, e.g., a desired reduction of angiogenesis and/or vasculogenesis. The precise desired therapeutic effect will vary according to the condition to be treated.

Included in the term “GPR124 antagonist”, without limitation, are antibodies, soluble GPR124 receptor ectodomains, both GPR124-derived and random peptides, nucleic acid aptamers and other binding moieties specific for GPR124; antisense, RNAi, siRNA, and other nucleic acids that specifically downregulate expression of GPR124; and small organic molecules that inhibit the activity of GPR124, e.g. by blocking binding or activation of the receptor.

Functional variants of the GPR124 polypeptide are of interest. Such variants may have substantial sequence similarity to a native GPR124 sequence, for example SEQ ID NO:1, usually at least about 90% sequence identity; at least about 95% sequence identity; up to at least about 99% sequence identity or more. Such variants may comprise 1, 2, 3, 4, 5, or more amino acid substitutions, deletions or additions, including conservative substitutions.

GPR124 peptides, which may be used in the methods of the invention, comprise at least about 10 amino acids, usually at least about 12 amino acids, at least about 15 amino acids, and which may include up to or more than 50 amino acids of a GPR124 peptide, including domains and larger fragments of about 100 amino acids or more; and modifications thereof, and may further include fusion polypeptides as known in the art in addition to the provided sequences. A combination of one or more forms may be used. The GPR124 sequence may be from any mammalian or avian species, e.g. primate sp., particularly humans; rodents, including mice, rats and hamsters; rabbits; equines, bovines, canines, felines; etc. Of particular interest are the human proteins.

Functional variants may also be assessed by the ability of a variant to activate pathways mediated by the wild-type GPR124 polypeptide, for example where the variant has an activity at least equal to the wild-type protein; and activity greater than the wild-type protein; or an activity not less than about 25% the activity of the wild-type protein. The activity may be ligand dependent or ligand independent, usually ligand dependent.

GPR124 has been identified as having certain activities, as reported herein, in the activation of angiogenesis, and such assays may be performed according to the examples set forth herein to determine activity of a GPR124 variant, and of compounds that modulate GPR124 activity.

By “isolated” is meant that the compound is separated from all or some of the components that accompany it in nature.

Macular Degeneration. Age-related macular degeneration is atrophy or degeneration of the macula. It is a common cause of worsening central vision in elderly patients. Funduscopic findings are diagnostic; fluorescein angiography assists in directing treatment. Treatment is with laser photocoagulation and low-vision devices.

Two different forms occur: In atrophic AMD (dry form), often referred to as geographic atrophy, there is irregular pigmentation of the macular region but no elevated macular scar and no hemorrhage or exudation in the macular region. In exudative AMD (wet or neovascular form), which is much less common, a subretinal network of choroidal neovascularization forms. This network is often associated with hyperpigmentation of the macula and soft drusen. A localized elevation of an area of the macula or a pigment epithelial detachment may be caused by hemorrhage or fluid accumulation. Eventually, this network leaves an elevated scar at the posterior pole.

Both forms of AMD are often bilateral and are preceded by development of drusen (small yellow deposits that form under the macula). In atrophic AMD, central visual acuity is lost slowly and painlessly. Rapid vision loss is more typical of exudative AMD. Although peripheral vision and color vision are generally unaffected, the patient may become legally blind in the affected eye(s).

AMD is diagnosed by clinical appearance of the retina. Fluorescein angiography may reveal a neovascular membrane beneath the retina. An angiogram is obtained when findings suggestive of neovascularization are present; such findings include subretinal hemorrhage, localized retinal elevation, retinal edema, and gray discoloration of the subretinal space. Fluorescein angiography demonstrates and characterizes a subretinal choroidal neovascular membrane.

If exudative AMD is untreated, vision typically deteriorates substantially, often to blindness. However, peripheral vision is usually retained. Results of treatment depend on the size, location, and type of neovascularization. Currently available treatment include thermal laser photocoagulation of neovascularization outside the fovea. Photodynamic therapy, a laser treatment, provides benefit under specific circumstances. Pegaptanib is an injectable selective vascular endothelial growth factor antagonist that can be used for the treatment of neovascular AMD. Other treatments being evaluated include transpupillary thermotherapy, subretinal surgery, and macular translocation surgery.

Diabetic Retinopathy. Diabetic retinopathy includes microaneurysms, hemorrhages, exudates, and macular edema occurring with diabetes of at least several years' duration. Vision rarely decreases until late in the disease. Diagnosis is by funduscopy; further details are elucidated by fluorescein angiography. Current treatment includes controlling diabetes and laser coagulation of threatening lesions.

Diabetic retinopathy is a major cause of blindness and tends to be particularly severe in type 1 diabetes. The degree of retinopathy is highly correlated with both duration of diabetes and poor blood glucose control. Nonproliferative retinopathy develops first. Proliferative retinopathy is more severe and may lead to vitreous hemorrhage and retinal detachment.

Proliferative retinopathy is characterized by abnormal new vessel formation (neovascularization), which occurs on the vitreous surface of the retina and may extend into the vitreous cavity and cause vitreous hemorrhages. Fibrous tissue that forms with the vessels may contract, resulting in retinal detachment. Neovascularization may also occur in the anterior segment of the eye on the iris, which can result in neovascular membrane growth in the angle of the eye at the peripheral margin of the iris, leading to neovascular glaucoma. Vision loss with proliferative retinopathy may be severe.

Proliferative retinopathy is diagnosed when fine preretinal capillaries are observed on the optic nerve or retinal surface. Retinal hemorrhage may develop in the vitreous cavity when these abnormal vessels are damaged. In extreme cases, retinal detachment may occur with white preretinal membranes forming over the retinal surface, especially over the major retinal vessels. Detachment and contraction of the vitreous gel contribute to retinal detachment by pulling the retina anteriorly from its attachments over the major vessels.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm or arteriovenous malformation, an angioma, blood dyscrasias, cardiac failure, cardic arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Stroke can be modeled in animals, such as the rat (for a review see Duverger et al. (1988) J Cereb Blood Flow Metab 8(4):449-61), by occluding certain cerebral arteries that prevent blood from flowing into particular regions of the brain, then releasing the occlusion and permitting blood to flow back into that region of the brain (reperfusion). These focal ischemia models are in contrast to global ischemia models where blood flow to the entire brain is blocked for a period of time prior to reperfusion. Certain regions of the brain are particularly sensitive to this type of ischemic insult. The precise region of the brain that is directly affected is dictated by the location of the blockage and duration of ischemia prior to reperfusion. One model for focal cerebral ischemia uses middle cerebral artery occlusion (MCAO) in rats. Studies in normotensive rats can produce a standardized and repeatable infarction. MCAO in the rat mimics the increase in plasma catecholamines, electrocardiographic changes, sympathetic nerve discharge, and myocytolysis seen in the human patient population.

The present methods are applicable to brain tumors, including glioblastoma. Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, usually cerebral tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are highly malignant, primitive tumors that arise in the posterior fossa, primarily in children. Meningiomas are common intracranial tumors that arise in the meninges and compress the underlying brain. Meningiomas are usually benign, but some “atypical” meningiomas may recur locally, and some meningiomas are frankly malignant and may invade the brain or metastasize. Atypical and malignant meningiomas are not as common as benign meningiomas. Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors depend mainly on the location in the brain and the size of the tumor. Since each area of the brain is responsible for specific functions, the symptoms will vary a great deal. Tumors in the frontal lobe of the brain may cause weakness and paralysis, mood disturbances, difficulty thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an GPR124 antagonist” includes a plurality of such antagonists and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention provides methods of modulating angiogenesis in an individual. The methods generally involve administering to an individual an effective amount of a GPR124 agonist or antagonist. The methods are useful to treat conditions and disorders associated with or resulting from angiogenesis, including pathological angiogenesis.

The results presented herein indicate that GPR124 antagonist are useful to treat conditions and disorders associated with and/or resulting from pathological angiogenesis, including, e.g., cancer, atherosclerosis, proliferative retinopathies, excessive fibrovascular proliferation as seen with chronic arthritis, psoriasis, and vascular malformations such as hemangiomas and arteiriovenous malformations.

The present invention includes methods of reducing angiogenesis in an individual. The methods generally involve administering to an individual an effective amount of a GPR124 antagonist.

The present invention also includes methods of increasing angiogenesis, particularly increasing angiogenesis in the CNS, by administering an agonist of GPR124.

GPR124 antagonists or agonists can be identified using readily available methods, including those described herein. The ability of a candidate agent to reduce angiogenesis can be assessed in vitro or in vivo using any known method, including, but not limited to, an in vitro Matrigel assay, disc- and plug-based angiogenesis systems, migration assays, proliferation assays, apoptosis assays, a murine model of hind limb ischemia, a murine model of cancer, animal models of retinal vascularization and the like. Assays directed at determining barrier activity, e.g. BBB activity, and expression of proteins involved in maintaining the BBB are also of interest. In some embodiments, an inhibitor of GPR124 is assessed fr its ability to downregulate expression of Glut1 in the brain.

Also included in screening methods are methods utilizing the activity of GPR124 in pericytes of the vasculature, where, for example, the effect of a candidate agent on pericytes expressing GPR124 may be assessed in vitro or in vivo. In vitro assays include contacting a candidate agent with a pericyte expressing GPR124, and determining the effect of the agent on an acitiy of pericytes, e.g. sensing the presence of angiogenic stimuli, depositing or degrading extracellular matrix and controlling endothelial cell proliferation and differentiation in a paracrine fashion. In vivo studies may assess the status of pericytes, e.g. by staining, immunohistochemistry, analysis of gene expression at the mRNA and/or ptoein level, etc. after the animal is contacted with a candidate agent.

Upon reading the present specification, the ordinarily skilled artisan will appreciate that the pharmaceutical compositions comprising a GPR124 antagonist described herein can be provided in a wide variety of formulations. More particularly, the GPR124 antagonist can be formulated into pharmaceutical compositions by combination with appropriate, pharmaceutically acceptable carriers or diluents, and may be formulated into preparations in solid, semi-solid (e.g., gel), liquid or gaseous forms, such as tablets, capsules, powders, granules, ointments, solutions, suppositories, injections, inhalants and aerosols.

The GPR124 antagonist formulation used will vary according to the condition or disease to be treated, the route of administration, the amount of GPR124 antagonist to be administered, and other variables that will be readily appreciated by the ordinarily skilled artisan. In general, and as discussed in more detail below, administration of GPR124 antagonists can be either systemic or local, and can be achieved in various ways, including, but not necessarily limited to, administration by a route that is oral, parenteral, intravenous, intra-arterial, inter-pericardial, intramuscular, intraperitoneal, intra-articular, intra-ocular, topical, transdermal, transcutaneous, subdermal, intradermal, intrapulmonary, etc.

In pharmaceutical dosage forms, the GPR124 antagonists may be administered in the form of their pharmaceutically acceptable salts, or they may also be used alone or in appropriate association, as well as in combination, with other pharmaceutically active compounds. The following methods and excipients are merely exemplary and are in no way limiting.

The GPR124 antagonist can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Formulations suitable for topical, transcutaneous, and transdermal administration may be similarly prepared through use of appropriate suspending agents, solubilizers, thickening agents, stabilizers, and preservatives. Topical formulations may be also utilized with a means to provide continuous administration, for example, incorporation into slow-release pellets or controlled-release patches.

The GPR124 antagonist can also be formulated in a biocompatible gel, which gel can be applied topically or implanted (e.g., to provide for sustained release of GPR124 antagonist at an internal treatment site). Suitable gels and methods for formulating a desired compound for delivery using the gel are well known in the art (see, e.g., U.S. Pat. Nos. 5,801,033; 5,827,937; 5,700,848; and MATRIGEL™).

For oral preparations, the GPR124 antagonist can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The GPR124 antagonist can be utilized in aerosol formulation to be administered via inhalation. The compounds of the present invention can be formulated into pressurized acceptable propellants such as dichlorodifluoromethane, propane, nitrogen and the like.

Furthermore, the GPR124 antagonist can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term unit dosage form, as used herein, refers to physically discrete units suitable as unitary dosages for human and/or animal subjects, each unit containing a predetermined quantity of GPR124 antagonist calculated in an amount sufficient to produce the desired reduction in angiogenesis in association with a pharmaceutically acceptable diluent, carrier or vehicle. The specifications for the unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Modulators of GPR124 may be targeted to pericytes for tissues outside of the CNS by formulation with a targeting moiety. A targeting moiety, as used herein, refers to all molecules capable of specifically binding to a particular target molecule and forming a bound complex. Thus the ligand and its corresponding target molecule form a specific binding pair.

Examples of targeting moieties include, but are not limited to antibodies, lymphokines, cytokines, receptor proteins such as CD4 and CD8, solubilized receptor proteins such as soluble CD4, hormones, growth factors, peptidomimetics, synthetic ligands, random peptides, nucleic acid aptamers and the like which specifically bind desired target cells, and nucleic acids which bind corresponding nucleic acids through base pair complementarity. Targeting moieties of particular interest include peptidomimetics, peptides, nucleic acid aptamers, antibodies and antibody fragments (e.g. the Fab′ fragment).

In some embodiments, a GPR124 antagonist is administered in a combination therapy with one or more additional therapeutic agents. Exemplary therapeutic agents include therapeutic agents used to treat cancer, atherosclerosis, proliferative retinopathies, chronic arthritis, psoriasis, hemangiomas, etc. Of particular interest are combinations with agents that inhibit the activity of VEGF and VEGF related pathways, e.g. SU11248; PTK787; and BAY 43-9006 are oral tyrosine kinase inhibitor that inhibit the VEGF receptors. Antibodies and soluble receptors of VEGF have also been tested in the clinic, e.g. the monoclonal antibody bevacizumab. Inhibition of VEGF-mediated calcineurin signaling by DSCR1 and DSCRL1 disrupts endothelial cell function and tumor angiogenesis. Pazopanib induces dose-dependent inhibition of VEGF-induced MM cell adhesion on HUVECs. Such agents may provide for additive or synergistic combinations with inhibitors of GPR124.

Suitable chemotherapeutic agents that may be combined with a GPR124 antagonist include, but are not limited to, the alkylating agents, e.g. Cisplatin, Cyclophosphamide, Altretamine; the DNA strand-breakage agents, such as Bleomycin; DNA topoisomerase II inhibitors, including intercalators, such as Amsacrine, Dactinomycin, Daunorubicin, Doxorubicin, Idarubicin, and Mitoxantrone; the nonintercalating topoisomerase II inhibitors such as, Etoposide and Teniposide; the DNA minor groove binder Plicamycin; alkylating agents, including nitrogen mustards such as Chlorambucil, Cyclophosphamide, Isofamide, Mechlorethamine, Melphalan, Uracil mustard; aziridines such as Thiotepa; methanesulfonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine; antimetabolites, including folate antagonists such as Methotrexate and trimetrexate; pyrimidine antagonists, such as Fluorouracil, Fluorodeoxyuridine, CB3717, Azacytidine, Cytarabine; Floxuridine purine antagonists including Mercaptopurine, 6-Thioguanine, Fludarabine, Pentostatin; sugar modified analogs include Cyctrabine, Fludarabine; ribonucleotide reductase inhibitors including hydroxyurea; Tubulin interactive agents including Vincristine Vinblastine, and Paclitaxel; adrenal corticosteroids such as Prednisone, Dexamethasone, Methylprednisolone, and Prodnisolone; hormonal blocking agents including estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone; and the like.

### Dose

The dose of GPR124 antagonist or agonist administered to a subject, particularly a human, in the context of the present invention should be sufficient to effect a therapeutic reduction or increase in angiogenesis in the subject over a reasonable time frame. The dose will be determined by, among other considerations, the potency of the particular GPR124 modulating agent employed and the condition of the subject, as well as the body weight of the subject to be treated. The size of the dose also will be determined by the existence, nature, and extent of any adverse side-effects that might accompany the administration of a particular compound.

In determining the effective amount of GPR124 modulating agent in the modulation of angiogenesis, the route of administration, the kinetics of the release system (e.g., pill, gel or other matrix), and the potency of the antagonist are considered so as to achieve the desired anti-angiogenic effect with minimal adverse side effects. The GPR124 modulating agent will typically be administered to the subject being treated for a time period ranging from a day to a few weeks, consistent with the clinical condition of the treated subject.

As will be readily apparent to the ordinarily skilled artisan, the dosage is adjusted for GPR124 modulating agent according to their potency and/or efficacy relative to a standard. A dose may be in the range of about 0.01 μg to 10 mg, given 1 to 20 times daily, and can be up to a total daily dose of about 0.1 μg to 100 mg. If applied topically, for the purpose of a systemic effect, the patch or cream would be designed to provide for systemic delivery of a dose in the range of about 0.01 μg to 10 mg. If injected for the purpose of a systemic effect, the matrix in which the GPR124 modulating agent is administered is designed to provide for a systemic delivery of a dose in the range of about 0.001 μg to 1 mg. If injected for the purpose of a local effect, the matrix is designed to release locally an amount of GPR124 modulating agent in the range of about 0.003 μg to 1 mg.

Regardless of the route of administration, the dose of GPR124 modulating agent can be administered over any appropriate time period, e.g., over the course of 1 to 24 hours, over one to several days, etc. Furthermore, multiple doses can be administered over a selected time period. A suitable dose can be administered in suitable subdoses per day, particularly in a prophylactic regimen. The precise treatment level will be dependent upon the response of the subject being treated.

### Reducing Angiogenesis In Vivo

The instant invention provides a method of reducing angiogenesis in a mammal. The method generally involves administering to a mammal a GPR124 antagonist in an amount effective to reduce angiogenesis. An effective amount of an GPR124 antagonist reduces angiogenesis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or more, when compared to an untreated (e.g., a placebo-treated) control.

Whether angiogenesis is reduced can be determined using any known method. Methods of determining an effect of an agent on angiogenesis are known in the art and include, but are not limited to, inhibition of neovascularization into implants impregnated with an angiogenic factor; inhibition of blood vessel growth in the cornea or anterior eye chamber; inhibition of endothelial cell proliferation, proliferation, migration or tube formation in vitro; modulation of retinal vascularization; the chick chorioallantoic membrane assay; the hamster cheek pouch assay; the polyvinyl alcohol sponge disk assay. Such assays are well known in the art and have been described in numerous publications, including, e.g., Auerbach et al. ((1991) Pharmac. Ther. 51:1-11), and references cited therein.

The invention further provides methods for treating a condition or disorder associated with or resulting from pathological angiogenesis. In the context of cancer therapy, a reduction in angiogenesis according to the methods of the invention effects a reduction in tumor size; and a reduction in tumor metastasis. Whether a reduction in tumor size is achieved can be determined, e.g., by measuring the size of the tumor, using standard imaging techniques. Whether metastasis is reduced can be determined using any known method. Methods to assess the effect of an agent on tumor size are well known, and include imaging techniques such as computerized tomography and magnetic resonance imaging.

### Conditions Amenable to Treatment

Any condition or disorder that is associated with or that results from pathological angiogenesis, or that is facilitated by neovascularization (e.g., a tumor that is dependent upon neovascularization), is amenable to treatment with a GPR124 antagonist.

Conditions and disorders amenable to treatment include, but are not limited to, cancer; atherosclerosis; proliferative retinopathies such as diabetic retinopathy, age-related maculopathy, retrolental fibroplasia; excessive fibrovascular proliferation as seen with chronic arthritis; psoriasis; and vascular malformations such as hemangiomas, and the like.

The instant methods are useful in the treatment of both primary and metastatic solid tumors, including carcinomas, sarcomas, leukemias, and lymphomas. Of particular interest is the treatment of CNS tumors. Thus, the methods are useful in the treatment of any neoplasm, including, but not limited to, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The instant methods are also useful for treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, the instant methods are useful for reducing metastases from the tumors described above either when used alone or in combination with radiotherapy and/or other chemotherapeutic agents.

Other conditions and disorders amenable to treatment using the methods of the instant invention include autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemangiomas, and capillary proliferation within atherosclerotic plaques; Osler-Webber Syndrome; plaque neovascularization; telangiectasia; hemophiliac joints; angiofibroma; and excessive wound granulation (keloids).

In some embodiments, an agent that modulates GPR124 is a small molecule, e.g., a small organic or inorganic compound having a molecular weight of more than about 50 daltons and less than about 20,000 daltons, e.g., from about 50 daltons to about 100 daltons, from about 100 daltons to about 200 daltons, from about 200 daltons to about 500 daltons, from about 500 daltons to about 1000 daltons, from about 1000 daltons to about 2500 daltons, from about 2500 daltons to about 5000 daltons, from about 5000 daltons to about 7,500 daltons, from about 7,500 daltons to about 10,000 daltons, from about 10,000 daltons to about 15,000 daltons, or from about 15,000 daltons to about 20,000 daltons. Agents may comprise functional groups necessary for structural interaction with proteins and/or nucleic acids, e.g., hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

In some embodiments, an agent that modulates GPR124 gene is a nucleic acid, e.g., an antisense RNA, an interfering RNA (including short interfering RNA; “siRNA”), a ribozyme, and the like, usually a nucleic acid encoding a GPR124 sequence, operably linked to a promoter active in the cells of interest. In other embodiments, nucleic acids can be used as aptamers which bind to the GPR124 molecule as functional agonists or antagonists.

Antisense oligonucleotides (ODN), include synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Among nucleic acid oligonucleotides are included phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Nucleic acid molecules of interest also include nucleic acid conjugates. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed.

Also of interest are RNAi agents. RNAi agents are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In other embodiments, a GPR124 modulating agent is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones, which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell). When antibody gene synthesis is induced in the transfected host, the heavy and light chain proteins self-assemble to produce active antibodies that can be detected by screening with the antigen or immunogen.

Antibodies with a reduced propensity to induce a violent or detrimental immune response in humans (such as anaphylactic shock), and which also exhibit a reduced propensity for priming an immune response which would prevent repeated dosage with the antibody therapeutic or imaging agent are preferred for use in the invention. Even through the brain is relatively isolated behind the blood brain barrier, an immune response still can occur in the form of increased leukocyte infiltration, and inflammation. Although some increased immune response against the tumor is desirable, the concurrent binding and inactivation of the therapeutic or imaging agent generally outweighs this benefit. Thus, humanized, single chain, chimeric, or human antibodies, which produce less of an immune response when administered to humans, are preferred for use in the present invention. Also included in the invention are multi-domain antibodies.

Antibody fragments that recognize specific epitopes may be generated by techniques well known in the field. These fragments include, without limitation, F(ab′)₂ fragments, which can be produced by pepsin digestion of the antibody molecule, and Fab fragments, which can be generated by reducing the disulfide bridges of the F(ab′)₂ fragments.

In one embodiment of the invention, a human or humanized antibody is provided, which specifically binds to the extracellular region of GPR124 target with high affinity. Binding of the antibody to the extracellular region can lead to receptor down regulation or decreased biological activity, and decrease in angiogenesis, invasion and/or decrease in tumor size.

Candidate anti-GPR124 target antibodies can be tested for by any suitable standard means, e.g. ELISA assays, proliferation assays, migration assays, etc. As a first screen, the antibodies may be tested for binding against the immunogen, or against the entire extracellular domain or protein. As a second screen, anti-GPR124 target candidates may be tested for binding to an appropriate cell line, or to primary tumor tissue samples. For these screens, the anti-GPR124 target candidate antibody may be labeled for detection.

In vivo models for human brain tumors, particularly nude mice/SCID mice model or rat models, have been described, for example see Antunes et al. (2000). J Histochem Cytochem 48, 847-58; Price et al. (1999) Clin Cancer Res 5, 845-54; and Senner et al. (2000). Acta Neuropathol (Berl) 99, 603-8. Once correct expression of the protein in the tumor model is verified, the effect of the candidate anti-protein antibodies on the tumor vasculature in these models can be evaluated, wherein the ability of the anti-protein antibody candidates to alter protein activity is indicated by a decrease in tumor growth or a reduction in the tumor vasculature.

### Stimulation of Therapeutic Angiogenesis

In some embodiments, a stimulator of therapeutic angiogenesis is administered to an individual in need thereof. In these embodiments, the stimulator of angiogenesis is an active agent that increases GPR124 activity or expression, and increases angiogenesis. Thus, in some embodiments, the instant invention provides a method of increasing or stimulating angiogenesis in a mammal. The method generally involves administering to a mammal an active agent in an amount effective to enhance GPR124 activity, thereby increasing angiogenesis.

An effective amount of an GPR124 activator increases angiogenesis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 2-fold, at least about 5-fold, at least about 10-fold, or more, when compared to an untreated (e.g., a placebo-treated) control. Stimulation of angiogenesis is useful to treat a variety of conditions that would benefit from stimulation of angiogenesis, stimulation of vasculogenesis, increased blood flow, and/or increased vascularity.

Examples of conditions and diseases amenable to treatment according to the method of the invention related to increasing angiogenesis include any condition associated with an obstruction of a blood vessel, e.g., obstruction of an artery, vein, or of a capillary system. Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like. Examples of conditions or diseases that can be prevented using the methods of the invention include, but are not necessarily limited to, heart attack (myocardial infarction) or other vascular death, stroke, death or loss of limbs associated with decreased blood flow, and the like.

### Screening Assays

The present invention further provides methods of identifying an agent that modulates angiogenesis. The methods may involve contacting a cell that is responsive to GPR124 with a test agent, for example in a competitive assay with GPR124; and assessing the effect of the test agent upon GPR124 mediated effects. Alternatively, an agonist or antagonist of GPR124 may be designed to bind to or mimic the activity of GPR124, e.g. in the design of a polypeptide or peptidomimetic agent. Such an agent may then be tested in any standard angiogenesis assay to confirm activity.

The terms “agent”, “substance”, and “compound” are used interchangeably herein, with the interchangeable terms “candidate agent,” and “test agent” referring to agents used in screening assays to identify those having a desired activity in modulating angiogenesis according to the present invention. “Agents” encompass numerous biological and chemical classes, including synthetic, semi-synthetic, or naturally-occurring inorganic or organic molecules, including synthetic, recombinant or naturally-occurring polypeptides and nucleic acids (e.g., nucleic acids encoding a gene product, antisense RNA, siRNA, and the like). “Candidate agents” or “test agents” particularly include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

In general, agents of interest include small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Agents, particularly candidate agents, are also found among biomolecules including peptides, saccharides, fatty acids, steroids, purines, pyrimidines, derivatives, structural analogs or combinations thereof.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### GPR124 (Tumor Endothelial Marker 5, TEM5)

GPR124, also known as tumor endothelial marker 5 (TEM5), encodes an orphan G-protein coupled receptor found to be specifically upregulated in tumor-associated endothelium. GPR124/TEM5a was one of the 46 genes identified through serial analysis of gene expression (SAGE) profiles of endothelium derived from normal and colorectal cancer tissue that was purported to be upregulated in tumor endothelium. Expression has been reported in primary colorectal cancer, endothelium of lung tumors, brain tumors and metastatic lesions of the liver. Expression of mouse GPR124/TEM5a in tumors was analyzed using mice implanted with B16 mouse melanoma and HCT116 human colon carcinoma cells. Murine GPR124 was significantly expressed in vessels penetrating both melanoma and colon carcinoma tumors.

GPR124 is predicted to encode a seven-pass transmembrane protein (1329 amino acids), characteristic of members of the G protein coupled receptor (GPCR) family. The amino terminal extracellular region, which is approximately 760 amino acids long, contains four simple leucine rich repeats (LRR), one carboxy-terminal type LRR, one immunoglobulin-type domain and one putative hormone-receptor domain followed by a GPCR proteolysis site (GPS). The hydrophobic domain of GPR124 shares homology with members of the secretin family of GPCRs (class II) while the LRR domain shares homology with LIG-1 and SLIT proteins. The mouse ortholog of human GPR124, identified by database search of mouse ESTs, shows an overall amino acid sequence identity of 88% and is most homologous at its LRR repeats and transmembrane domains, suggesting functional conservation.

### Results

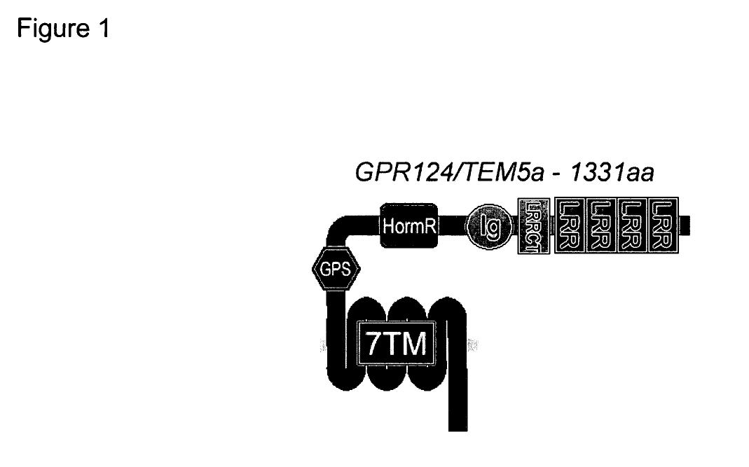

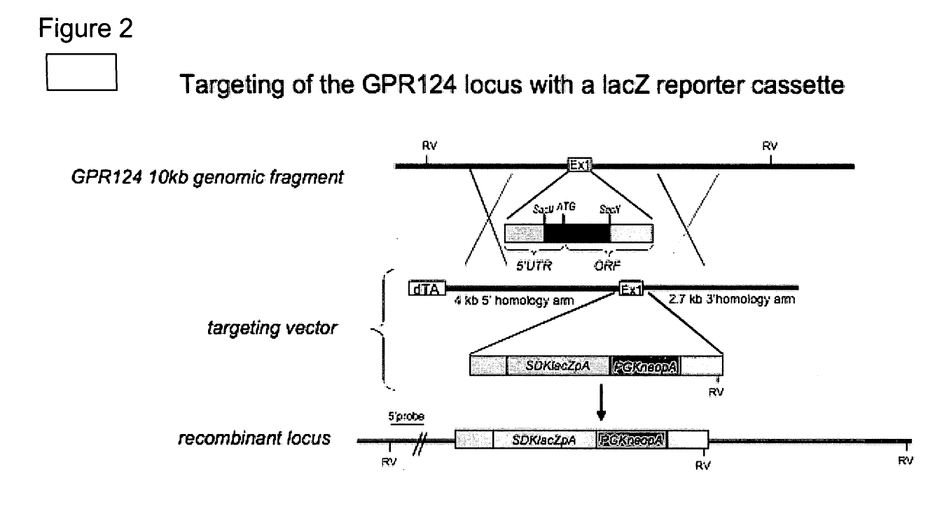

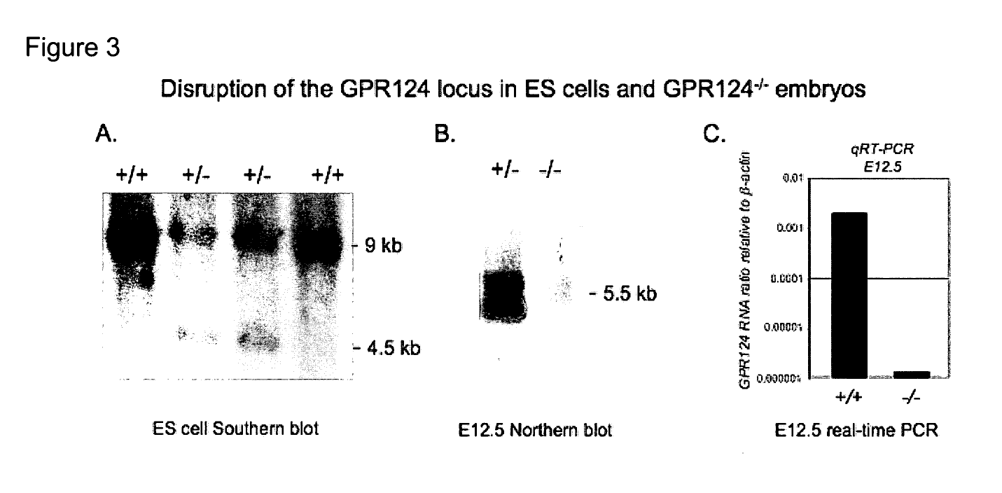

Generation of GPR124/TEM5 knockout mice. GPR124/TEM5 is an orphan G-protein coupled receptor with a large ectodomain (FIG. 1) that has previously noted to be expressed in tumor endothelium. Despite this expression pattern, the actual function of GPR124 in angiogenic regulation had not been previously established, since the receptor is orphan, and since gene deletion studies had not been performed. To identify the functional relationship of GPR124 to angiogenesis we produced mice in which the GPR124/TEM5 locus was inactivated by replacement of a region of exon 1 with lacZ, to both disrupt gene function as well as allow visualization of TEM5 expression by histochemical p-galactosidase staining. A clone encoding the murine GPR124 genomic locus was isolated from a 129sV BAC library. From this clone, a targeting construct was produced containing a 4.0 kb 5′ homology arm and a 2.7 kb 3′ arm, and in which a SacII fragment of exon 1 (including the start codon) was replaced with a lacZ reporter (SDKlacZpA) and a neomycin selection cassette (PGKneopA) (FIG. 2). This construct was linearized and electroporated into mouse ES cells. Out of 100 ES cell clones screened, two correctly targeted clones were identified by Southern blot analysis (FIG. 3a) and injected into blastocysts for generation of chimeric mice. Chimeric mice derived from both ES cell clones transmitted the mutant allele successfully through the germline, and absence of GPR124RNA in homozygous embryos was confirmed by Northern blot (FIG. 3b). Absence of GPR124 mRNA was also confirmed by real-time PCR, demonstrating ˜1000-fold decrease in GPR124RNA (FIG. 3c) and absence of protein in knockout was demonstrated using polyclonal antisera which we have generated against the C-terminus of TEM5a (described in subsequent sections).

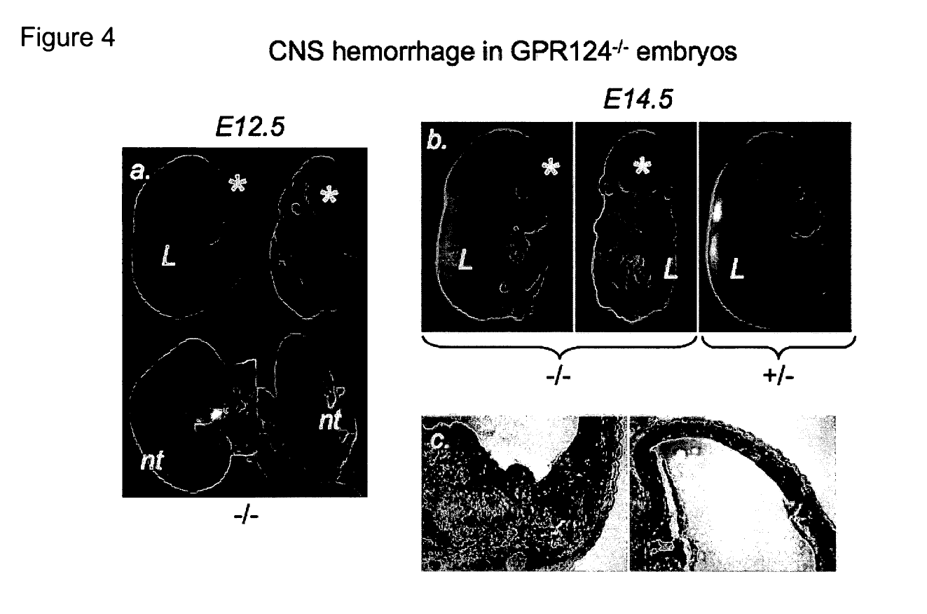

Embryonic lethality in homozygous GPR124^{-/-} animals. Homozygous GPR124^{-/-} mice, in which both copies of the GPR124 gene were disrupted, exhibited prominent hemorrhage in utero, leading to embryonic lethality by E15.5 (FIG. 4). Detailed analysis revealed the presence of bilateral hemorrhage, particularly the forebrain in telencephalon and ganglionic eminences, with 100% penetrance. The first evidence of hemorrhage was observable at E11.5. which invariably, originated in the ganglionic eminence of the forebrain, and was accompanied by some degree of cavitation and dissociation of the neuroepithelium. Subsequently, hemorrhage spread throughout the forebrain, with the eventual appearance of gross bleeding in the ventricle and occasional local extension to other regions of the brain. Additional hemorrhage was present transiently in the neural tube (FIG. 4). Extensive evaluation did not reveal hemorrhage in sites other than the central nervous system. Liveborn homozygous mutant offspring have not been detected among nearly 200 mice generated from heterozygous intercrosses.

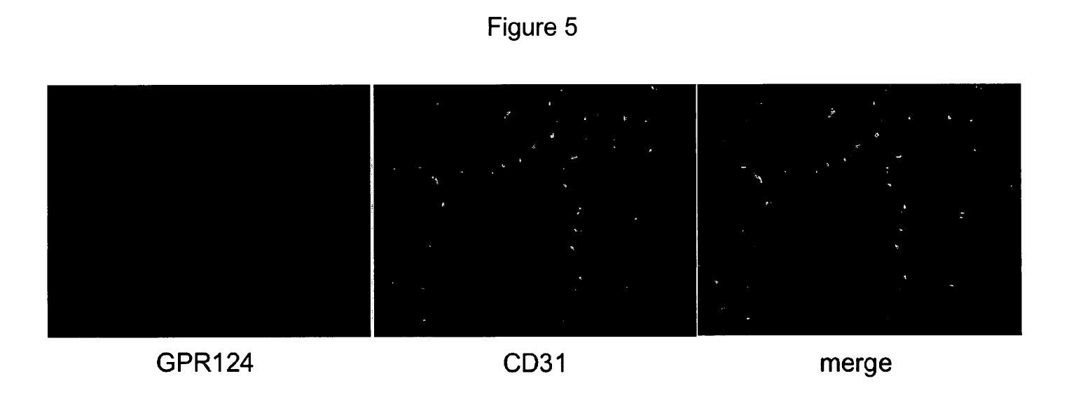

GPR124 mutation produces severe deficits in angiogenic sprouting and migration. Using a polyclonal antibody raised against the ectodomain of GPR124, we noted strong expression extending through the entire microvascular network of the embryonic brain (FIG. 5). The vasculature of the embryonic brain forms initially by vasculogenesis to form the perineural plexus, which resides at the pial surface. A subsequent wave of angiogenesis then occurs, as sprouts derived from the perineural plexus invade the neuroepithelium, undergoing extensive arborization as they migrate towards the ventricular surface. Notably, this angiogenesis first occurs in the ganglionic eminence, and the pial-to-ventricular direction of migration is opposite to the direction of migration of nascent neurons.

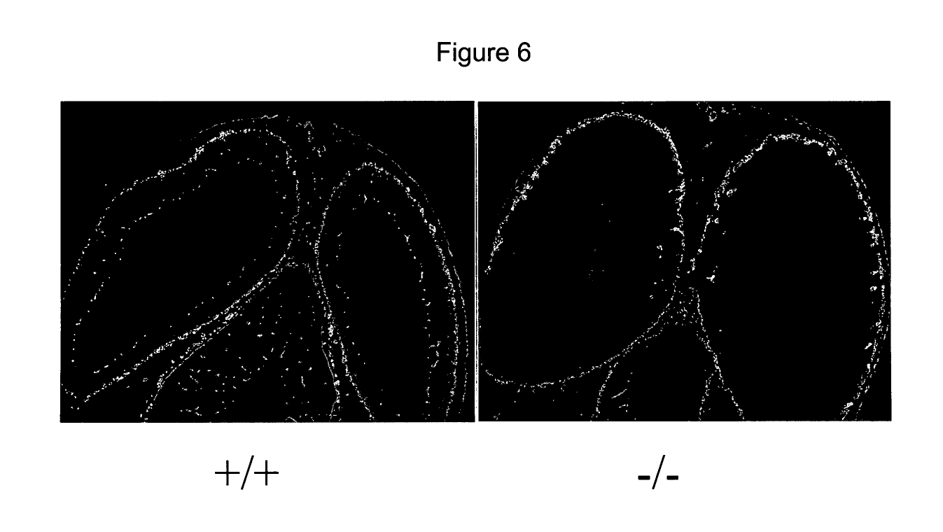

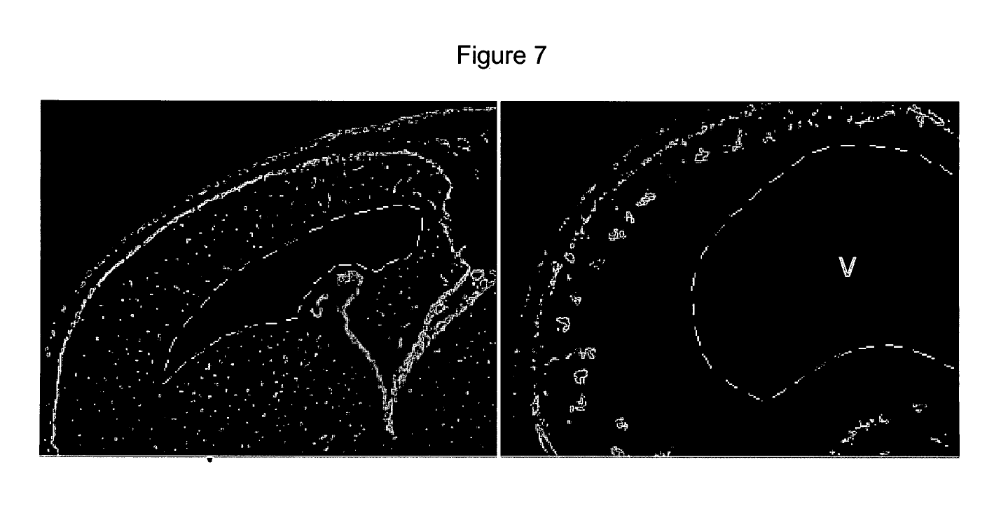

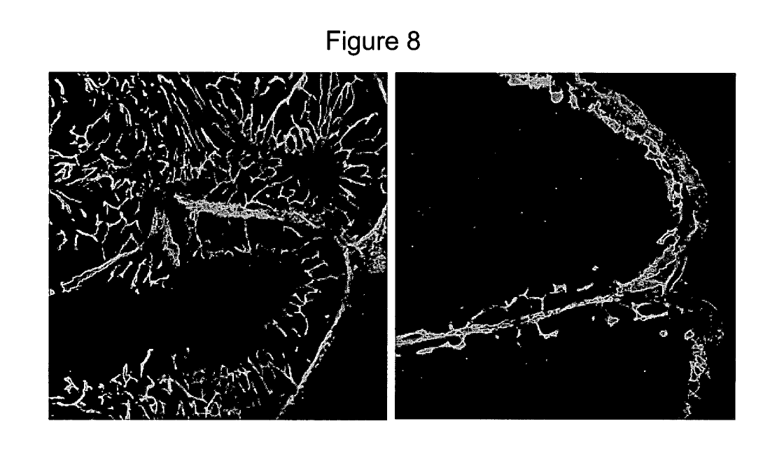

We analyzed the CNS microvasculature of GPR124^{-/-} mice using laminin antibodies previously used to delineate CNS angiogenesis. Examination of microvessels using anti-ZO-1, anti-laminin, or anti-collagen IV all revealed profound deficits in angiogenic sprouting and migration at E12.5 (FIG. 6), E14.5 (FIG. 7), or E15.5 (FIG. 8). E12.5 wild-type animals exhibited a homogenous distribution of puntate microvessels throughout the neuroepithelium (FIG. 6). However, the microvessels of GPR124^{-/-} penetrated poorly into the neuroepithelium and did not migrate to the periventricular area, resulting in large unvascularized areas (FIG. 6, see boxed areas for reference). GPR124 knockout animals at E14.5 exhibited similar migratory deficits, with large unvascularized areas (FIG. 7); however these migratory defects were accompanied by the development of large glomeruloid vascular malformations resulting from the inability of endothelial cells to sprout radially towards the ventricles. Similar phenotypes were also observed at E15.5 (FIG. 8). These data unequivocally demonstrate that GPR124 functions as a pro-angiogenic receptor, since loss-of-function produces a marked impairment of angiogenesis. Accordingly, pharmacologic inhibition of GPR124 is useful for anti-angiogenic therapies.

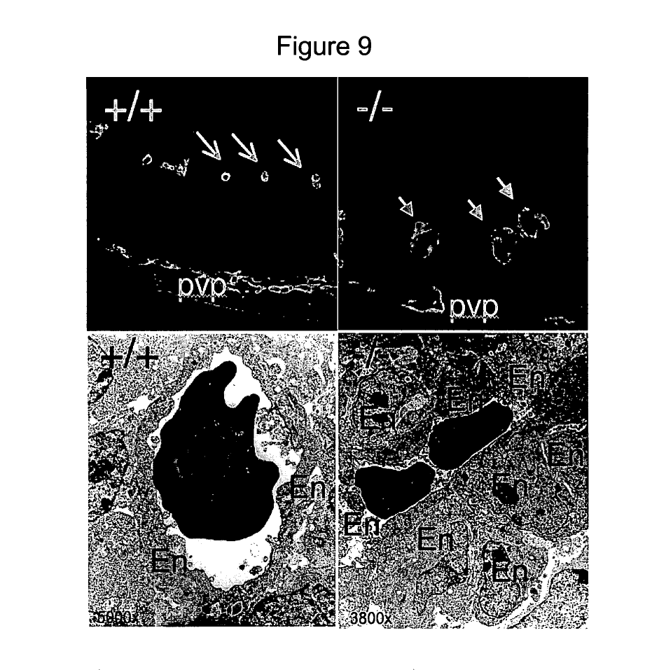

Ultrastructure of GPR124-deficient vasculature. Detailed examination of the structure of the malformations indicated the presence of both endothelial cells and pericytes (FIG. 9, top). Further, electron microscopy indicated an extremely haphazard cellular architecture with numerous abnormal endothelial cells associating with each other rather than organizing around a lumen (FIG. 9, bottom). Similarly, endothelium in the glomeruloid aggregates exhibited pronounced loss of cytoplasmic extension, suggesting that GPR124 exerts critical roles in maintaining the cytoskeleton (FIG. 9, bottom). The vascular lesions in knockout mice also expressed the arterial marker NRP1 as well as the embryonic venous markers EphB4 and VEGFR3, consistent with an arteriovenous malformation. GPR124 ko vascular malformations express both arterial and venous markers. Immunofluorescence analysis of vascular malformations in E12.5 GPR124 knockout brain (telencephalon) revealed expression of markers of embryonic arterial (NRP1) and venous (EphB4, VEGFR3) endothelium.

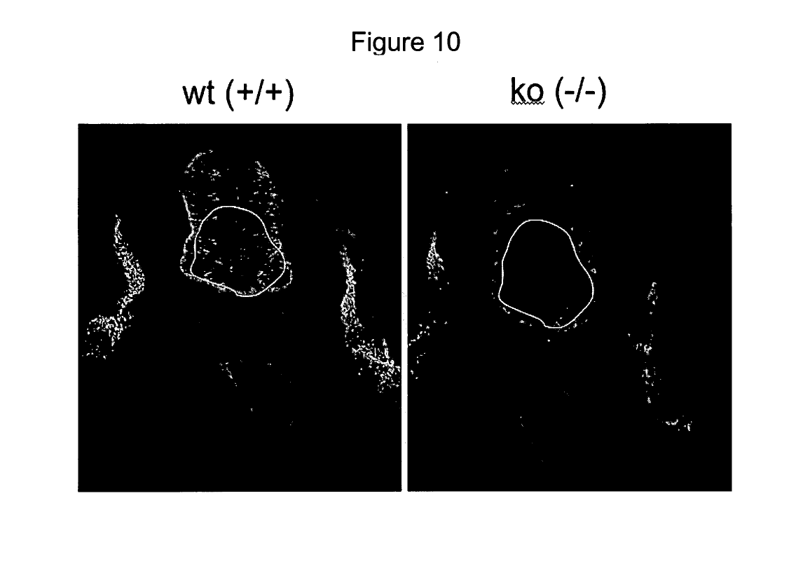

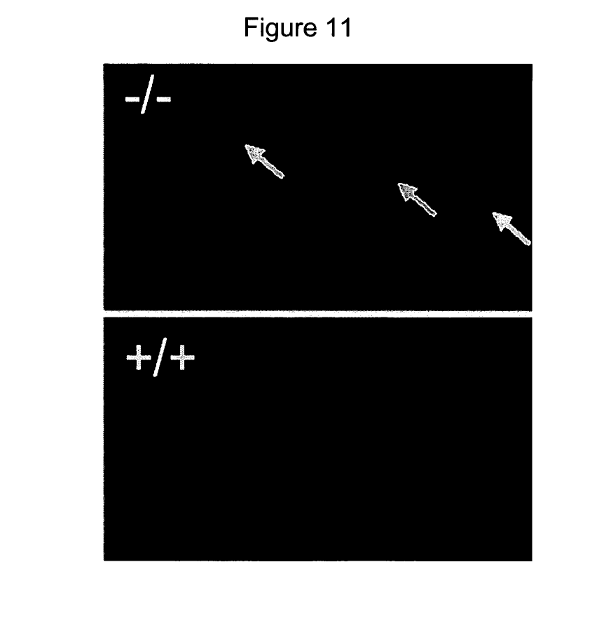

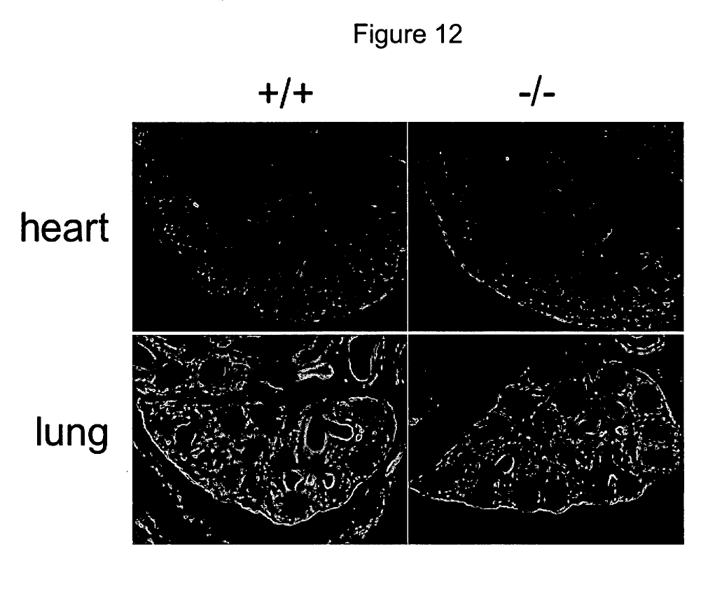

Angiogenic deficits in GPR124-deficient mice are confined to the CNS. Endothelial migratory deficits were also present in neural tube, as the endothelium in GPR124 knockout animals exhibited delayed migration into the ventral spinal cord (FIG. 10). This was accompanied by the presence of glomeruloid malformations similar to in brain (FIG. 11). Within the brain, the angiogenic defects were confined to the telencephalon which later forms the cerebral hemispheres, and were not present in diencephalon, midbrain or hindbrain. However, in contrast to the CNS angiogenic deficits in brain and neural tube, angiogenesis in non-CNS organs was unaffected, for instance in heart and lung (FIG. 12). This strong tropism for the CNS stands in marked contrast to endothelial receptor systems such as VEGF/VEGFR, Angiopoietin/Tie2, EphinB2/EphB4 and Notch/DLL4, which exert more pleitrophic effects on angiogenesis. Consequently, GPR124 represents the first example of an endothelial receptor selectively regulating CNS angiogenesis, with implications for therapeutic applications for the brain and spinal cord.

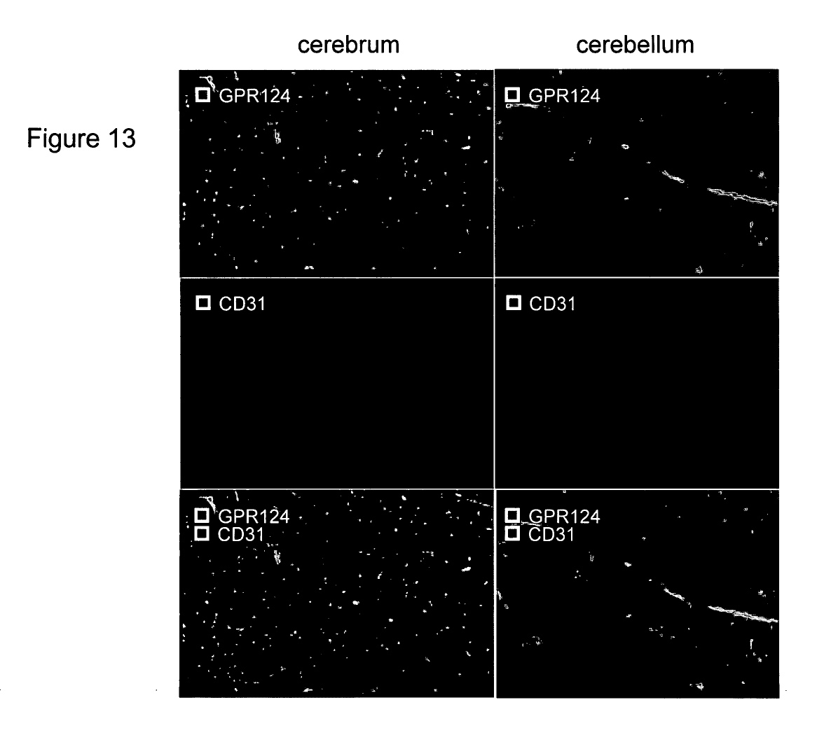

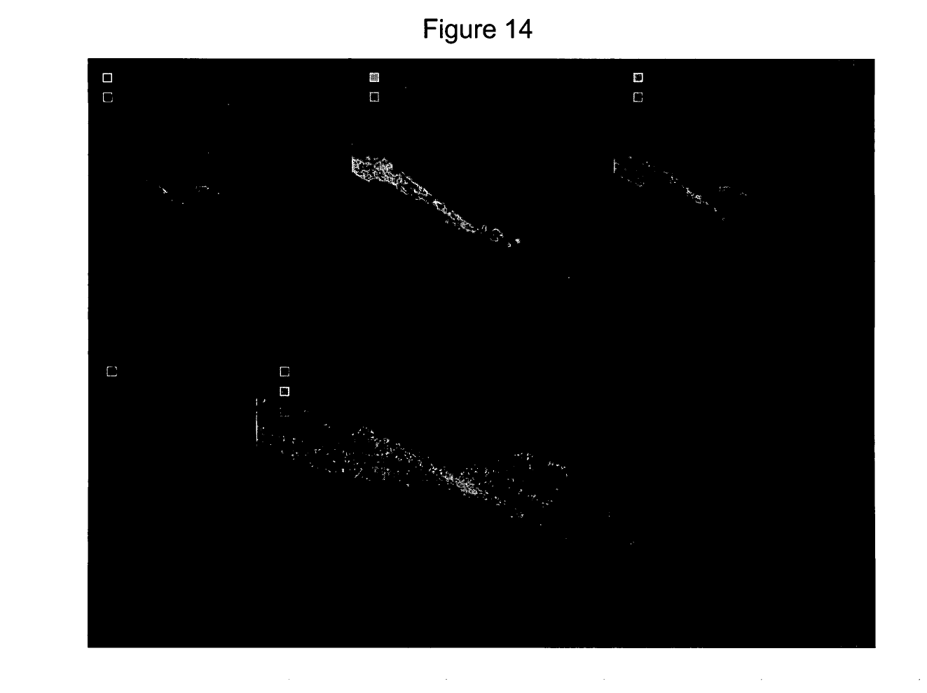

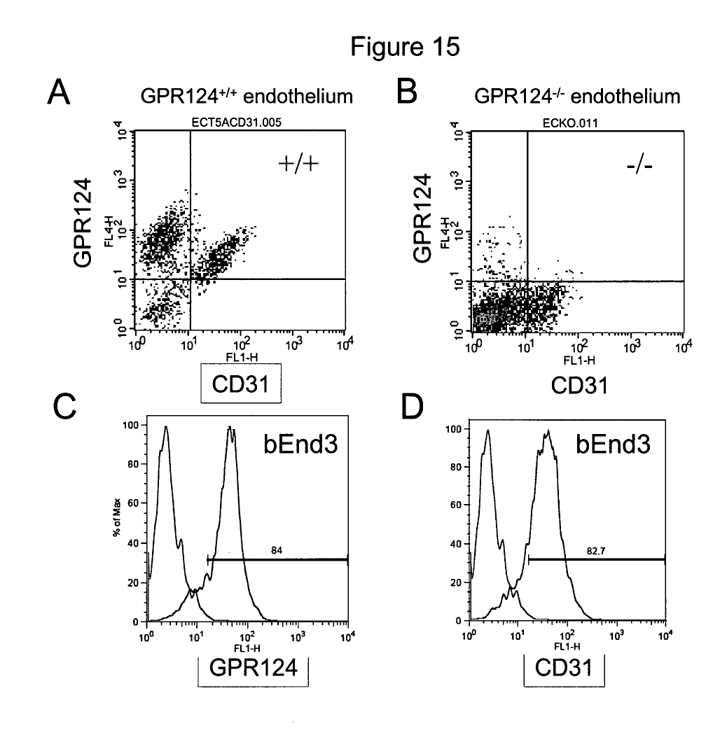

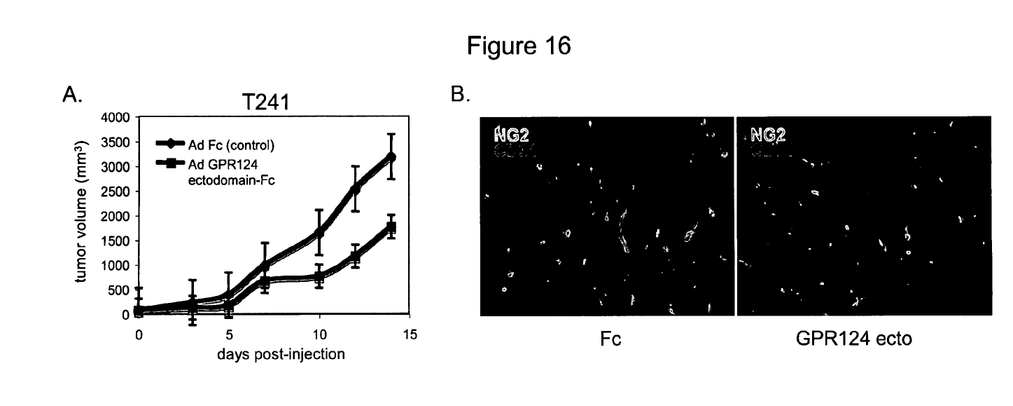

GPR124 expression in CNS endothelial cells and pericytes. Towards understanding the medical contexts in which GPR124 inhibition could be useful, we performed a detailed analysis of adult GPR124 expression patterns. In the adult brain, immunofluorescence analysis revealed pan-endothelial GPR124 expression with expression in all capillary beds as well as larger vessels, in a manner identical to the endothelial marker CD31 (FIG. 13). Detailed examination by high-resolution confocal microscopy, however, revealed additional expression in brain pericytes (FIG. 14). We further confirmed the presence of GPR124 on brain endothelium by FACS using a polyclonal GPR124 antibody. Accordingly, CD31⁺ brain endothelium from E12.5 mouse quantitatively expressed cell-surface GPR124 by FACS, as did the brain endothelial cell line bEND3 (FIG. 15). In contrast, brain endothelium from GPR124 knockout mice as well as HUVEC isolated from human umbilical vein were GPR124-negative by FACS (FIG. 16).

GPR124 expression in CNS endothelial cells and pericytes. The retina is contiguous with and is an extension of the central nervous system. As numerous angiogenesis-dependent disorders affect the retina, such as macular degeneration and diabetic retinopathy, we examined GPR124 expression in retinal vasculature. This revealed abundant GPR124 expression in all retinal microvasculature, in both endothelial and pericyte compartments. GPR124 is expressed in both endothelial cells and pericytes of the adult retina. Frozen flat mount preparations of C57Bl/6 adult mouse retina were examined for expression of GPR124, CD31 and PDGFRβ by immunofluorescence. Strong co-localization was observed between GPR124 and CD31 (endothelial marker), yielding a signal indicative of endothelial GPR124 expression. Additionally, co-localization is observed between GPR124 and PDGFRβ (pericyte marker). The robust expression of GPR124 alone in retinal vasculature, combined with our identification of GPR124 as a pro-angiogenic receptor, strongly indicates the potential utility of GPR124 inhibition for the over-vascularization that is pathogenic for macular degeneration and diabetic retinopathy.

GPR124 expression in non-CNS organs is largely confined to pericytes. In all adult organs examined, whether CNS or non-CNS, GPR124 expression was exclusively vascular. However, while GPR124 was expressed in the vasculature of non-CNS organs such as kidney, pancreas, spleen, lung and liver, this expression was generally in pericytes, not endothelial cells, with the exception of the liver, which has no pericytes. Organs that did not express GPR124 in the microvasculature but had occasional expression in large vessels included heart, muscle and intestine. The pronounced restriction of endothelial GPR124 expression to CNS within adult organs further supports the concept of CNS-selective angiogenic regulation by this receptor. Expression staining showed GPR124 expression in pericytes, not endothelial cells, of kidney and pancreas. Kidney and pancreas represent non-CNS organs which abundantly express GPR124 in their vasculature. However, as opposed to brain, the kidney and pancreas express GPR124 in pericytes, not endothelial cells. Further, in both kidney and pancreas, the signal from GPR124/PDGFRβ was clearly excluded from the CD31 (endothelial) signal.

GPR124 is expressed in tumor pericytes, not tumor endothelium in subcutaneous models. GPR124 was initially designated as Tumor Endothelial Marker 5, reflecting its presumed expression in tumor endothelium. Using our affinity-purified anti-GPR124 polyclonal sera, we examined GPR124 expression in tumors with particular attention to endothelial versus pericyte expression. Unexpectedly, we noted GPR124 expression in pericytes, not endothelial cells, of numerous human and mouse tumors subcutaneously and orthotopically implanted in mice, including B16 melanoma, 4T1 mammary carcinoma, and T241 fibrosarcoma. Expression in tumor pericytes suggests the use of GPR124 inhibition for anti-angiogenic therapy directed against tumor pericytes, and call into question the initial designation of GPR124 as Tumor Endothelial Marker 5. Given the expression of GPR124 in brain endothelium, GPR124-targeted anti-angiogenic therapies might be most efficacious in brain tumors. GPR124 is expressed in pericytes, not endothelial cells, of B16 melanoma and 4T1 mammary carcinoma. Frozen sections of subcutaneously implanted B16 melanoma or 4T1 mammary carcinoma were analyzed for expression of GPR124 and CD31 by immunofluorescence. CD31 (endothelial marker) and GPR124 are clearly expressed in different cell populations indicating that GPR124 is not expressed in tumor endothelium in these models. Frozen sections of subcutaneously implanted 4T1 mammary carcinoma were analyzed for expression of PDGFRβ (pericyte marker) and GPR124. The merge of these two signals gives a completely co-localizing signal (bottom right panel) indicating the predominant expression of GPR124 in pericytes, not endothelium, of 4T1 mammary carcinoma.

GPR124 is expressed in pericytes, not endothelial cells, of T241 fibrosarcoma. Frozen sections of subcutaneously implanted T241 fibrosarcoma were analyzed for expression of GPR124, CD31 and PDGFRβ by immunofluorescence. T241 tumors express GPR124 in pericytes, not endothelial cells as evidenced by the presence of a signal, indicating the merge of the GPR124 and the pericyte marker PDGFRβ). The signal is clearly excluded from the CD31 (endothelial) signal, again consistent with pericyte expression. GPR124 is further expressed in an additional unidentified stromal element in the tumor as indicated by isolated cells.

GPR124 is expressed in pericytes of the corpus luteum. The corpus luteum was examined as a second site of adult neo-angiogenesis in addition to tumor. Again, similar to tumors and non-CNS organs, GPR124 was expressed in pericytes, not endothelium, of the corpus luteum of C57Bl/6 following hormonal stimulation. Strikingly, receptor expression was not detected in unstimulated ovary, confirming GPR124 induction during active angiogenesis. GPR124 is expressed in pericytes, not endothelial cells, of the corpus luteum. Ovulation was induced by PMSG and hCG treatment of female C57Bl/6 mice. Four days later, frozen sections of ovaries were analyzed for expression of GPR124, CD31 and PDGFRβ by immunofluorescence. Co-localization is not observed between GPR124 and CD31 (endothelial marker). By contrast, co-localization is observed between GPR124 and PDGFRβ (pericyte marker), as evidenced by a merged signal. This indicates that GPR124 is expressed in pericytes, not endothelial cells, of the corpus luteum.

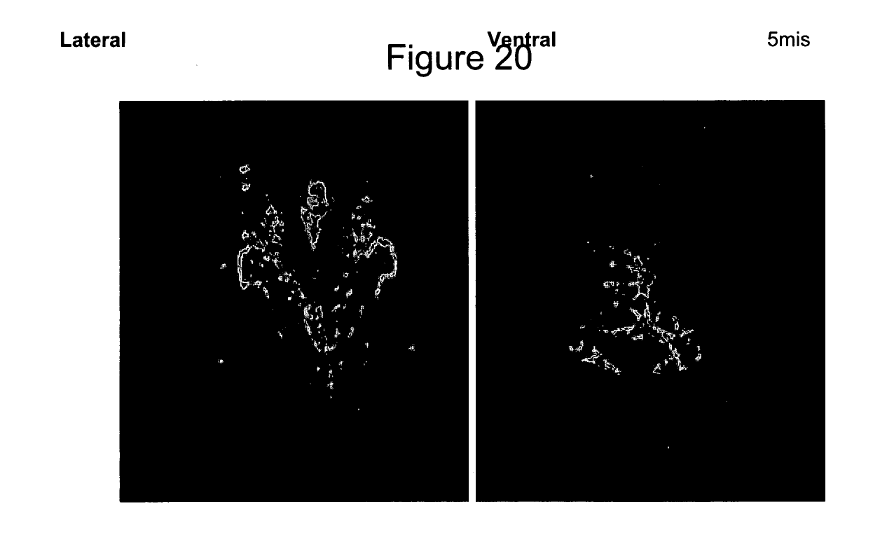

Inhibition of tumor growth and angiogenesis by a GPR124 ectodomain. We previously described the ability of expression of the GPR124 extracellular domain to elicit a similar vascular phenotype as GPR124 gene knockdown in zebrafish, suggesting that the ectodomain is necessary and sufficient to bind the as-yet unidentified ligand. By analogy, the ability of GPR124 ectodomain to elicit anti-angiogenic effects in a tumor model was examined. Adult C57Bl/6 mice bearing subcutatenously implanted T241 fibrosarcomas received injection of adenovirus expressing either a GPR124 ectodomain-Fc fusion or a control Fc fragment. Tumor growth was significantly reduced (FIG. 16a), and immunofluorescence analysis indicated a 60% reduction in tumor pericyte content (FIG. 16b). The effects on tumor pericytes are completely consistent with the known pericyte expression of GPR124 in the T241 model (FIG. 20), and indicate the potential utility of GPR124 inhibitors such as ectodomains, or potentially antibodies or small molecules, for anti-angiogenic therapy of cancer.

VEGF expression is unaltered in GPR124-deficient mice. Angiogenesis pathways independent of VEGF are of great interest because of their application for therapy of tumors which are resistant to VEGF inhibition. We have shown in zebrafish that VEGF inhibition does not suppress GPR124 inhibition, suggesting independence of these two pathways. The reciprocal relationship was examined in GPR124 knockout mice. Notably, GPR124 inactivation in knockout mice did not affect VEGF-A expression, further attesting to the concept of GPR124 as an angiogenic pathway independent of VEGF. GPR124 gene deletion does not affect VEGF-A expression. Analysis of VEGF-A expression by immunofluorescence staining of transverse sections of E12.5 embryos was tested in a GPR124 knockout (−/−) or wild-type (+/+). VEGF-A expression was unaltered by GPR124 gene deletion, indicating that GPR124 function is independent of VEGF-A.

Overall, these studies provide a functional analysis of GPR124/TEM5. The current data, utilizing GPR124 gene disruption in mice, unequivocally demonstrate a pro-angiogenic function for GPR124, and reveal an unsuspected role for GPR124 as an essential regulator of central nervous system angiogenesis. The highly selective effects of GPR124 on CNS vascularization, combined with the highly restricted expression of GPR124 in CNS endothelial cells including brain and retina, suggests that GPR124 is an attractive candidate to mediate vascular bed-specific angiogenesis in neural tissues. Such tropism has particular implications for the use of GPR124 inhibition during CNS tumorigenesis, macular degeneration and diabetic retinopathy. Our findings that GPR124 acts independently of the VEGF pathway and that GPR124 ectodomains can act as GPR124 inhibitors and elicit anti-tumor and anti-angiogenic effects are consistent with this use. Conversely, the pro-angiogenic function of GPR124 can be harnessed to stimulate CNS angiogenesis, for instance for therapy of stroke.

In contrast to endothelial GPR124 expression in the CNS, GPR124 is largely expressed in pericytes in other organ systems as well as non-CNS sites of neo-angiogenesis including the corpus luteum and subcutaneously implanted tumors. These findings provide an unanticipated function of GPR124 in the regulation of pericyte function and call into question the initial description of GPR124 as a Tumor Endothelial Marker. The function of GPR124 in pericytes provides for therapeutic manipulation of this receptor in pericyte-directed anti-angiogenic therapy.

### Methods

Production of GPR124 knockout mice. We produced mice in which the GPR124/TEM5 locus was inactivated by replacement of a region of exon 1 with lacZ, to both disrupt gene function as well as allow visualization of TEM5 expression by histochemical β-galactosidase staining. A clone encoding the murine GPR124 genomic locus was isolated from a 129sV BAC library. From this clone, a targeting construct was produced containing a 4.0 kb 5′ homology arm and a 2.7 kb 3′ arm, and in which a SacII fragment of exon 1 (including the start codon) was replaced with a lacZ reporter (SDKlacZpA) and a neomycin selection cassette (PGKneopA). This construct was linearized and electroporated into mouse ES cells. Out of 100 ES cell clones screened, two correctly targeted clones were identified by Southern blot analysis and injected into blastocysts for generation of chimeric mice. Chimeric mice derived from both ES cell clones transmitted the mutant allele successfully through the germline, Confirmation of gene disruption was additionally obtained by Western blot as well as quantitative RT-PCR.

Production of rabbit-anti mouse GPR124 polyclonal antisera. A murine GPR124 ectodomain-Fc fusion containing all five leucine-rich repeats, the HormR and Ig domains was expressed in the conditioned medium of 293T cells by adenoviral infection in serum-free medium. The ectodomain fusion was used as immunogen in rabbits. Polyclonal anti-GPR124 sera were depleted of anti-Fc reactivity using sequential purification over IgG2a Fc columns, and the flow-through was affinity purified over GPR124-Fc-protein A agarose. The monoreactivity of this reagent was confirmed by lack of signal upon immunofluorescence staining of frozen sections from GPR124 knockout mice as well as lack of FACS signal on GPR124-knockout endothelial cells.

FACS analysis of GPR124 expression. E12.5 brain endothelial cells (EBEC) were isolated from wild-type or GPR124 knockout mice by collagenase digestion of brain tissue followed by magnetic bead isolation with anti-CD31 antibody. EBEC were cultured for 2 days and subsequently analyzed for expression of GPR124 and CD31 by FACS. Brain endothelial cell line BEnd3 cells were stained with polyclonal rabbit anti-GPR124 or monoclonal rat anti-CD31 antibodies. Staining was visualized by incubation with the appropriate Alexa 488-conjugated secondary antibodies followed by FACS analysis. Staining with secondary antibodies alone served as control.

Whole-mount retinal preparation. Eyes were dissected out of perfused adult C57Bl/6 mice (see below), fixed overnight in 1% PFA and stored in PBS for up to three weeks. Retinas were dissected out of the eyes and stained as floating sections in 24 well plates. Following staining, retinas were flat-mounted onto Superfrost/plus slides (Fisher) with vectashield, coverslipped, and imaged.

Immunofluorescence staining. Frozen sections from embryos, adult tissues, tumors or ovaries were prepared. For adult tissues, tumors or ovaries animals were anesthetized with Avertin and then perfused with 1% PFA/PBS through the aorta for two minutes before tissues were harvested. Tissues were fixed for one hour in 1% PFA/PBS on ice, rinsed in PBS and cryoprotected in 30% sucrose/PBS overnight at 4° C. The following day, tissues were embedded in OCT and frozen on dry ice. Embryo samples were prepared similarly except without perfusion. Cryostat sections (10 or 60 mm) were cut and mounted on Superfrost/plus slides. After drying for several hours, tissues were permeabilized by immersion in 0.3% TritonX-100/PBS (PBST). Slides were then incubated for one hour in 5% normal goat serum in PBST to block nonspecific antibody binding. Sections were double or triple-stained by overnight incubation at 4° C. in humidified chambers with affinity purified rabbit anti-mouse GPR124 antibody (see above) (1:500), hamster anti-mouse CD31 antibody (Chemicon 1:400) and rat anti-mouseCD140b/PDGFRβ (eBiosciences, 1:400). The following day, slides were washed with PBST several times and incubated in appropriate secondary antibodies conjugated to FITC, Cy3 or Cy5 (Jackson ImmunoResearch, 1:200) for one hour (10 mm sections) or four hours (60 mm sections). Slides were then washed with PBST and fixed in 4% PFA for five minutes. After brief washes in PBST and PBS, slides were mounted in Vectashield (Vector Laboratories) and imaged.

Confocal immunofluorescence microscopy. A Leica TCS SP2 AOBS microscope in the Stanford Cell Imaging Core was utilized. Z-stack reconstruction was performed on the confocal images using the Leica software.

Adenovirus production. GPR124 ectodomain-Fc fusion cDNA with the LRRx5, Ig and HormR domain was cloned in frame with a mouse IgG2α Fc immunoglobulin fragment and inserted into the E1 region of E1⁻E3⁻ Ad strain 5 by homologous recombination, followed by Ad production in 293 cells and CsCl gradient purification of virus. The construction of Ad Fc encoding IgG2αFc have been described previously (Kuo et al, 2001).

Inhibition of tumor growth and angiogenesis by systemic adenoviral delivery of GPR124 ectodomain. C57Bl/6 mice bearing pre-established T241 fibrosarcoma (n=10, 55 mm³) received single iv injection of 5×10⁸ pfu of adenovirus expressing a GPR124 ectodomain-Fc fusion protein or a control immunoglobulin IgG2a Fc fragment. Plasma transgene expression was confirmed by Western blotting. Tumor size in (mm³) was calculated from caliper measurements obtained over a 10-25 day period using the formula for a spheroid approximation 4/3×π length (mm)/2×(width (mm)/2)², with width as the smaller dimension.

For analysis of tumor vascularity, parallel cohorts of tumors were allowed to grow until 100-200 mm3 (8-12 days) after which they were resected and processed for immunofluorescence analysis. Tumors were harvested seven days post adenovirus injection and processed for immunofluorescence analysis. Vessels were stained with antibodies against CD31 for endothelial cells and NG2 (Chemicon, 1:200) for pericytes. Endothelial cell and pericyte content was quantified using Volocity software (Improvision).

Corpus luteum angiogeneisis model. Three to four week-old female C57Bl/6 mice were first injected i.p. with 5 IU of pregnant mare serum gonadotrophin (PMSG, Sigma). Two days later, ovulation was induced by treatment with 5 IU of human choriogonadotropin (hCG, Sigma). 48 hours after hCG injection, ovaries were harvested and processed for hematoxylin and eosin histological analysis according to standard protocols. Immunofluorescence analysis was performed as described above.

### Example 2

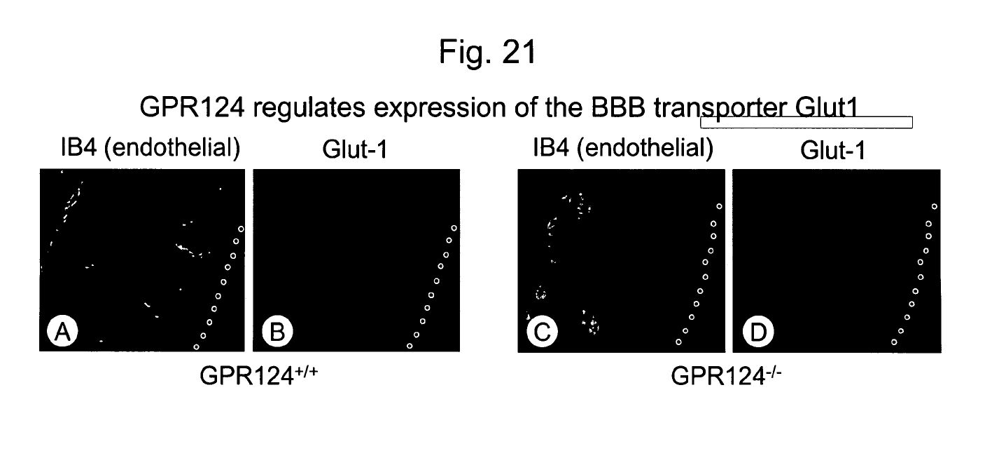

The pan-endothelial expression of GPR124 in brain vasculature suggests that this receptor could participate in regulation of the blood-brain barrier (BBB). We have found that GPR124 ko vasculature has a striking down-regulation of the Glut1 glucose transporter (FIG. 21), which is the major glucose transporter of the brain endothelium. This reveals for the first time a long-sought downstream target of GPR124 signaling and indicate that GPR124 can regulate BBB transporters such as Glut1.

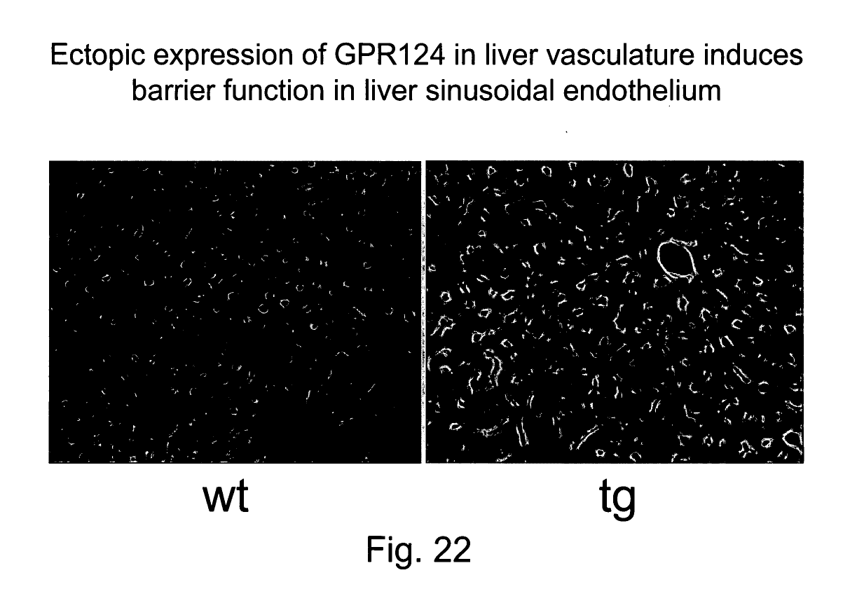

Conversely, it was found that ectopic expression of GPR124 in liver vasculature results in induction of barrier function in liver sinusoidal endothelium. We generated transgenic Tie2-GPR124 mice in which GPR124 is overexpressed in throughout the vasculature, both in the CNS and in other vascular beds. The hepatic vasculature of wild type mice was inherently leaky as indicated by tracer leakage resulting in obscurement of the sinusoidal vascular pattern (FIG. 22, left panel). However, in transgenic Tie2-GPR124 mice overexpressing in the liver vasculature, barrier properties were induced, resulting in markedly enhanced retention of the tracer within the sinusoids, and allowing the sinusoids to be clearly visualized (FIG. 22, right panel).

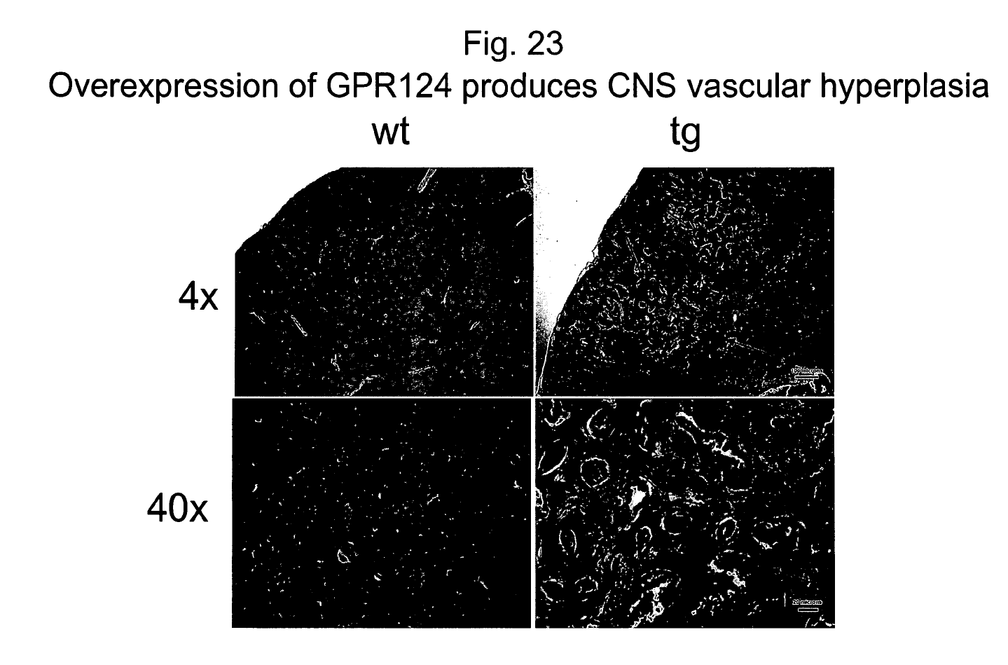

These same Tie2-GPR124 transgenic mice have allowed analysis of the effects of receptor overexpression on CNS angiogenesis, complementing our loss-of-function analysis in knockout mice. Examination of the brains of adult Tie2-GPR124 transgenic mice indicate foci of hyperplastic CNS vasculature engorged with red blood cells which was observed in transgenic (FIG. 23, right panels) but not wild-type (FIG. 23, left panels). These data indicate that GPR124 functions as a pro-angiogenic receptor, in agreement with the angiogenic deficits observed in knockout mice. These results further indicate that activation of GPR124 can be used to induce CNS angiogenesis.

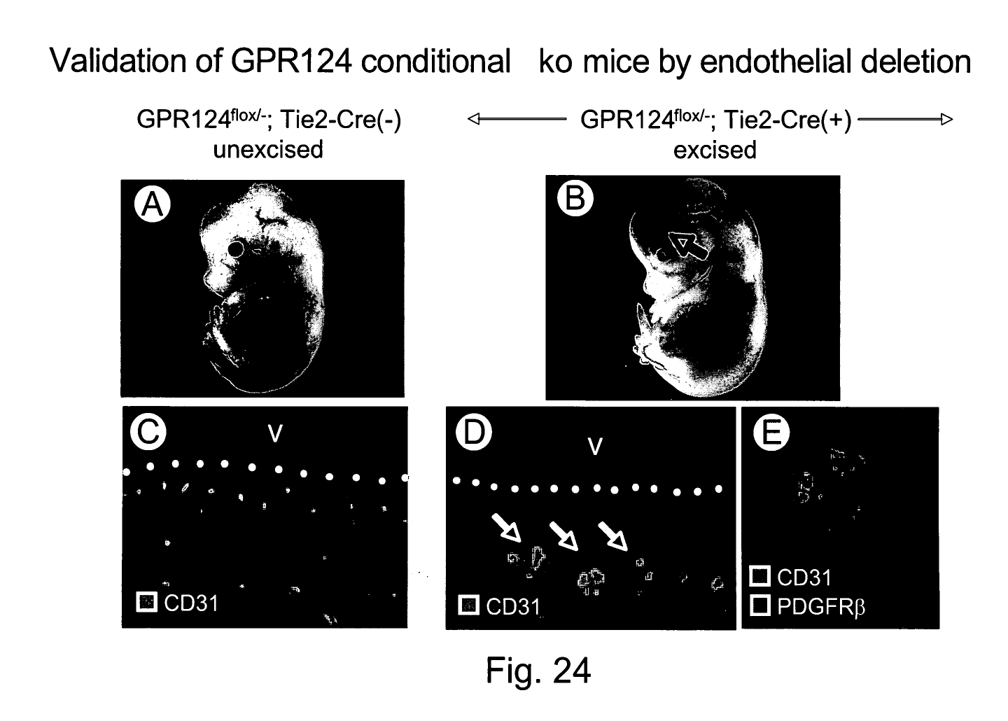

To confirm the importance of expression of GPR124 on endothelial cells to modulate CNS angiogenesis, we created GPR124 conditional knockout mice allowing Cre-mediated gene deletion. The endothelial-specific GPR124 deletion in GPR124flox/−Tie2-Cre(+) embryos was sufficient to fully phenocopy and recapitulate both the forebrain hemorrhage and CNS endothelial migration deficits of GPR124−/−ko mice, with peripherally located glomeruoid malformations (FIG. 24). These results further emphasize the critical role of GPR124 as a receptor expressed by CNS endothelium that stimulates CNS angiogenesis.

### Example 3

A zebrafish homolog of GPR124 exists and provides an alternative model system for studying the gene's role in vertebrate vascular development.

Zebrafish provide an excellent alternative to the mouse for functional genomics studies. Zebrafish embryos are externally fertilized, allowing immediate accessibility for experimentation and embryogenesis occurs entirely outside of the mother's body. Additionally, embryonic, vascular development is very rapid. Embryos receive sufficient oxygen to develop for several days in the absence of a functional vascular network, permitting functional characterization of genes that impair cardiovascular development. Importantly, zebrafish embryos are transparent, allowing easy visualization of fundamental vertebrate developmental processes. Finally, the relative ease with which gene function can be inhibited using morpholino anti-sense oligonucleotides, make zebrafish an ideal system for studying GPR124.

The following describes cloning of the zgpr124 gene, its expression during development, and its functional characterization. Embryos microinjected with zGPR124 antisense morpholino oligonucleotides exhibited vascular defects, specifically abnormal patterning of aortic arch arteries. Additionally, alcian blue staining indicated defects in cartilage formation within the pharyngeal arches of GPR124 morphants. Structure-function analysis of this orphan GPCR, which is not possible in mammalian systems, was performed by injecting embryos with GPR124 ectodomain mRNA. Ectodomain injections partially phenocopied morpholino injections, suggesting that GPR124 signaling occurs through ligand binding of the ectodomain. GPR124 expression was examined in embryos injected with VEGF-A morpholino and found to be unaltered compared to wild-type embryos, suggesting that GPR124 activity is not downstream of VEGF signaling. These findings reveal an essential role for GPR124 in zebrafish vascular development and pharyngeal arch patterning and suggest that GPR124 signaling is mediated through ligand binding of the ectodomain.

### Materials and Methods

### Zebrafish Stocks and Reagents

Zebrafish were grown and maintained at 28.5° C. WTAB strain (ZIRC) and Tg(flk1:EGFP) (kindly provided by DY.R. Stainier) were used. Embryos were collected from natural breedings, raised at 28.5° C. and staged.

Cloning of Full Length Gpr124. An incomplete zGPR124 ORF was generated by reverse transcription-PCR on total RNA isolated from 24 hpf embryos using primers designed to known zgpr124 sequence. This sequence was extended at the 5′ end by using the First Choice RLM-RACE kit (Ambion) according to the manufacturer's protocol.

Whole-mount In Situ Hybridization. Wild-type embryos were grown in 1-phenyl-2-thiourea (0.0002%) to prevent pigment development, dechorionated, fixed overnight in 4% paraformaldehyde/PBS (4% PFA/PBS) and stored in 100% methanol until use. Whole-mount in situ hybridizations were carried out as previously described. Previously published plasmids were used for both VE-cadherin and crestin riboprobe synthesis. Gpr124 antisense-probe was generated with RNA polymerase by using linearized vector containing 1.8 kb of gpr124 fragment.

Antisense Morpholino and mRNA injections. Morpholinos were synthesized by GeneTools LLC (Philomath, Oreg.). Knockdown of gpr124 was achieved by injection of specific morpholinos (MOs) into two-cell stage embryos. The sequences for morpholinos used are as follows: gpr124 2/3MO (splicing antisense), 5′_ACTGGAGAGGTCACTGCGCAGATTA; gpr124 ATG-MO (translational blocking antisense), 5′ GGATGCGGACGCAGGGTCCCGACAT; 5-mismatch MO, 5′ ACTcGAcAGGTgACTGCaCAGATTA. VEGF-A morpholino has been previously published.

cDNA fragments encoding full length zebrafish gpr124 ectodomain and ectodomain deletion mutants were subcloned into pCS2+ vector. Deletion mutants were generated by PCR overlap extension. mRNAs were synthesized in vitro using the SP6 mMessage mMachine System (Ambion) and 150-300 pg of capped mRNAs were injected into one-cell stage embryos.

Injected embryos were fixed in 4% PFA/PBS overnight at 4° C. and embedded in 1.5% agarose. Confocal microscopy was performed using a Leica. Images were rendered using Image J software.

### Results

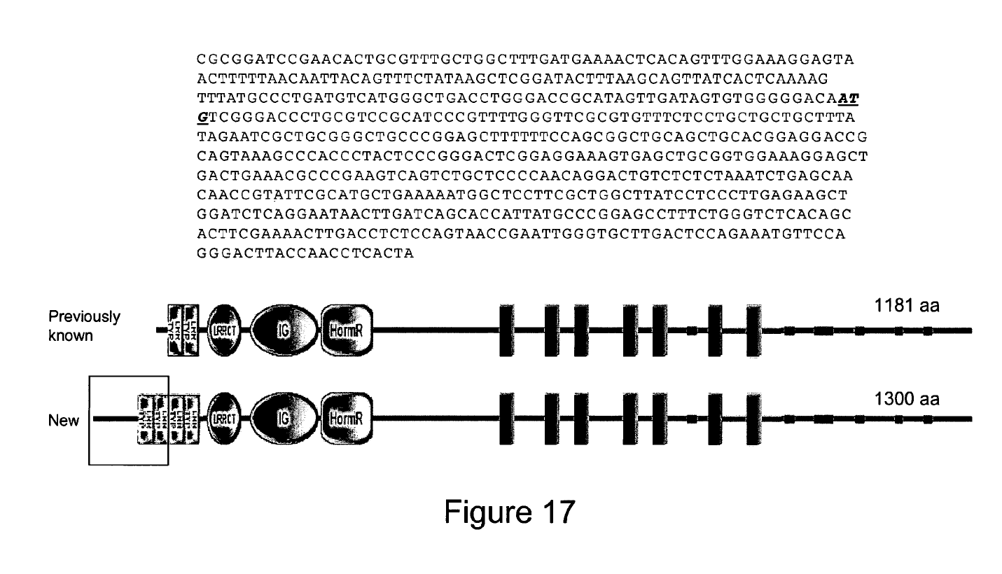

Cloning of full length zebrafish gpr124. The previously undeposited 5′ end of the zebrafish gpr124 homolog has been cloned by 5′ RACE (rapid amplification of cDNA ends), allowing subsequent cloning of full length zebrafish gpr124. 5′RACE extended the zebrafish sequence by 193 aa. The extended zebrafish gpr124 ORF encodes a protein of 1367 aa which is 49% identical to murine gpr124 and 54% identical to human gpr124. Like its murine and human homologs, zebrafish gpr124 encodes a 7-pass transmembrane protein with a large N-terminal extracellular region which is comprised of five LRRs, one immunoglobulin-type domain (IgG), one putative hormone-receptor domain (HR) and one GPCR proteolysis site (GPS) (FIG. 17).

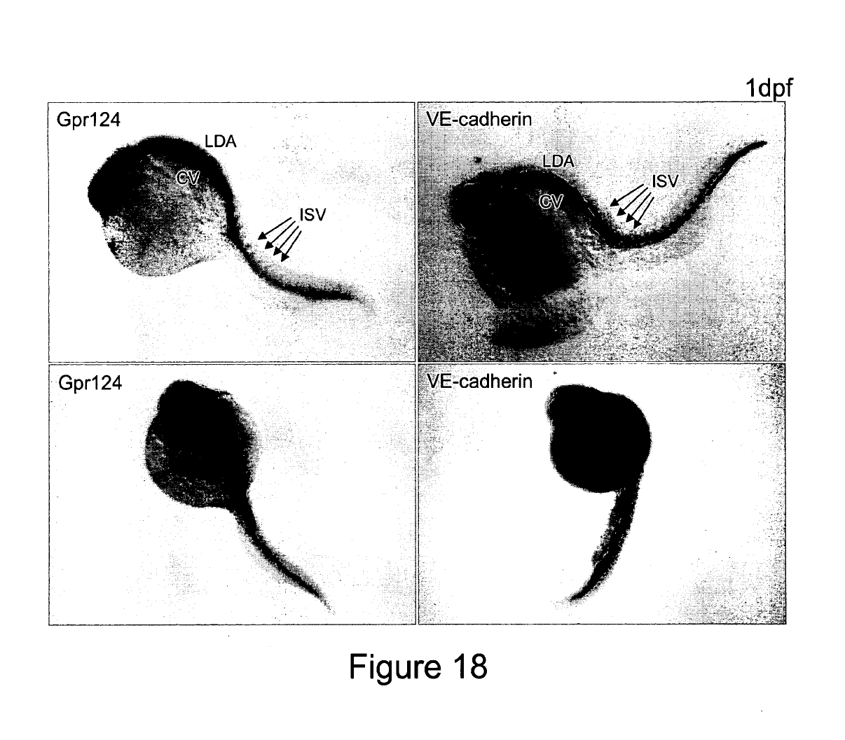

Expression of gpr124 in zebrafish. The expression pattern of zgpr124 during embryonic and early larval development was examined by whole mount in situ hybridization. Vascular expression of gpr124 becomes apparent at 25 somites, when gpr124 is detected in the axial vessels of the trunk and tail−the lateral dorsal aortae (LDA) and caudal vein (CV). In 1.5 dpf embryos, gpr124 is expressed not only in the lateral dorsal aorta and axial vein, but also in the sprouting intersegmental vessels (ISV). gpr124 is also detected in the cerebral vasculature at this stage, in the middle cerebral vein, as well as the primordial midbrain channel. In all of the developmental stages examined, gpr124 expression closely resembles the expression pattern of VE-cadherin, which is specifically expressed in the vascular endothelial cells in both developing tissue and mature vasculature {Larson, 2004 #43}. At 3 and 4 dpf, gpr124 expression is detected in the head vasculature and the pharyngeal arches, in a pattern similar to VE-cadherin expression in the aortic arch arteries (FIG. 18).

Role of gpr124 in zebrafish vascular development. The vascular expression pattern of gpr124 as well as the vascular phenotype observed in gpr124 knockout mice, suggest a potential role of gpr124 in zebrafish vascular development. Gpr124 gene function during zebrafish embryonic development was examined by gene specific morpholino antisense knockdown. We designed two MOs-one splice MO spanning intron2-exon3 (2/3 MO), which disrupts mRNA splicing and introduces a premature stop codon, and one translational start MO (ATG MO), which inhibits translation initiation. The ATG MO spans the start codon, inhibiting translation. The 1/2 MO spans the intron 1-exon 2 junction, causing intron retention and an ectopic stop codon. The 5mis MO has 5 mismatches (lower case) as opposed to the 1/2 MO and serves as a negative control MO. Sequences were as follows:

| | |
|---|---|
| ATG MO | 5′_GGATGCGGACGCAGGGTCCCGACAT |
| ½ MO | 5′_ACTGGAGAGGTCACTGCGCAGATTA |
| 5mis MO | 5′_ACTcGAcAGGTgACTGCaCAcATTA |

In addition, we designed a splice mismatch MO (5mis MO) to control for non-specific effects. Efficacy of the splice MO was confirmed by RT-PCR across flanking exons of the targeted splice acceptor site. A shift from a 246 by band in 5mis MO injected and wild-type uninjected embryos to a 5208 by band in 2/3MO injected embryos indicated retention of intron 2 in 2/3 MO injected embryos. Intron retention in 2/3 MO injected embryos introduced a premature stop codon, which resulted in a truncated protein terminating after the second LRR within the ectodomain.

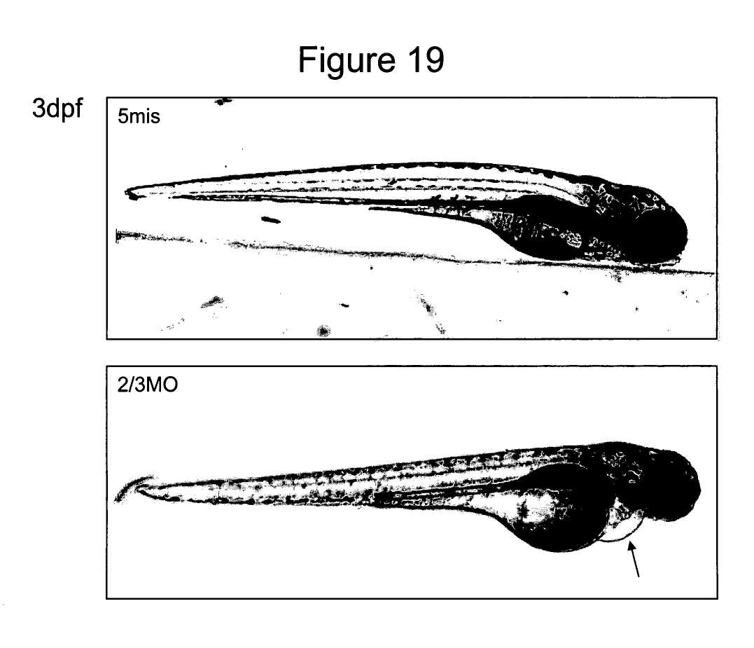

At 3 dpf, pericardial edema was observed in MO injected embryos, followed by yolk sac edema at 5 dpf (FIG. 19). To examine the effects of gpr124 knockdown on vascular development, we injected the MOs into Tg(flk1:EGFP) zebrafish. These transgenic fish express green fluorescent protein specifically in vascular endothelial cells and exhibit fluorescent vessels throughout development, allowing live imaging of the vasculature. Zebrafish vascular development begins ˜12 hpf when angioblasts migrate from the lateral plate mesoderm to form the major axial vessels—the dorsal aorta and posterior cardinal vein. MO injected embryos displayed normal vasculogenesis, as exhibited by normal formation of the axial vessels. Intersegmental vessels, which begin to sprout 24 hpf, were unaffected at low dose, while some missing ISVs were detected at the high dose at which curly and kinked tails were also observed. At 3 dpf, MO injected embryos began to exhibit angiogenic deficits in the head. MO injected embryos exhibited grossly abnormal aortic arch arteries, with a pronounced lack of anterior extension of AA1 as well as lack of lateral extension of the more posterior arch arteries (FIG. 20). Zebrafish have six aortic arch arteries, which are present by 2.5 dpf. AA1, also known as the mandibular arch, becomes greatly modified during development, elongating rostrally and eventually becoming the internal carotid artery providing the extracranial major blood supply to the head. AA3 and 4 primarily supply the cranial vasculature, while AA5 and 6 supply the trunk and tail vasculature. In addition to the aortic arch artery malformations, a noticeable decrease in complexity of the head vasculature was observed in morpholino injected embryos. A noticeable decrease in complexity of the head vasculature was observed in 1/2 MO morpholino injected Flk1-GFP zebrafish embryos as opposed to the 5 mis negative control morpholino.

The specificity of the 2/3 splice MO knockdown was confirmed using a second morpholino directed against the translational start site (ATG MO). The ATG MO produced a similar aortic arch artery phenotype as the 2/3 MO, which was not observed in embryos injected with the 5mis control MO. Non-overlapping morpholinos can produce a synergistic effect when co-injected and using two MOs together at a lower dose reduces the likelihood of mistargeting. To further control for specificity of the morpholino knockdown, we co-injected the 2/3 and ATG MOs at lower doses which individually have no effect. The co-injected embryos exhibited a similar aortic arch artery phenotype as embryos with single high dose MO injections, demonstrating synergy between the two morpholinos.

Structure function analysis of gpr124 ectodomain. Because gpr124 is an orphan receptor with an unknown ligand, structure function analysis in mammalian systems or in vitro is not possible. Without knowledge of ligand or receptor agonist, the receptor cannot be activated, and without knowledge of the downstream signaling pathway, there is no readout for receptor activation. The genetic accessibility of zebrafish embryos and the rapidity of development make zebrafish an ideal system for in vivo structure function analysis in the absence of a known ligand.

Gpr124 is a member of the family B or class II family of G-protein coupled receptors, specifically the large N-terminal family B seven transmembrane receptors. The large ectodomains in these receptors are not only important for ligand binding, but can alone form high affinity binding sites for the ligand, as has been previously shown with vasoactive intestinal peptide and adenylate cyclase activating polypeptide receptors. Because gpr124 is a member of the large N-terminal family, we next examined whether the GPR124 ectodomain is important for ligand binding.

Structure function analysis of gpr124 was performed using in vitro transcribed gpr124 ectodomain RNA. Gpr124 ectodomain was injected into embryos to determine whether the ectodomain was necessary and sufficient to bind ligand and could phenocopy the morpholino knockdown. This ectodomain was denoted LRRHR for its incorporation of the 5 leucine-rich repeats, the Ig domain and the HormR domain. Gpr124 ectodomain appeared to partially phenocopy the morpholino knockdown with angiogenic deficits in the aortic arch arteries. The GPR124 ectodomain produced a similar phenotype as the 1/2 splice MO, indicating the potential use of GPR124 ectodomain as a receptor antagonist. RNA encoding either was encoding the 5 leucine rich repeats, the Ig domain and the HormR domain of the GPR124 ectodomain (zLRRHR) or the 1/2 splice MO were injected into Flk1-GFP zebrafish embryos, followed by whole mount immunofluorescence to evaluate the head vasculature. The zLRRHR ectodomain appeared to partially phenocopy the 1/2 splice MO in the absence of rostral extension of head vasculature. While ectodomain-injected embryos displayed the similar lack of anterior extension of AA1, an additional widening of the body axis was observed, in which the more posterior arch arteries were extended further laterally. The partial phenocopy of ectodomain injections suggests that GPR124 signaling is mediated by ectodomain binding of the ligand and suggest that GPR124 ectodomain could be therapeutically useful as a GPR124 antagonist.

Epistatic analysis of gpr124 and VEGF. VEGF is an essential vascular specific growth factor, and remains one of the most critical drivers of vascular formation. VEGF is required both during the formation of immature vessels, as well as during the sprouting and remodeling of primitive vessels into a mature vascular network. As VEGF signaling has profound effects throughout angiogenesis, we next performed epistasis analysis to investigate whether gpr124 functions in the VEGF signaling pathway. Classical epistasis analysis is used to determine the order of genes in pathways, and can also establish whether a protein acts within a given pathway. Zebrafish embryos were injected with VEGF-A morpholino antisense oligonucleotides at a concentration which resulted in pericardial edema as well as missing intersegmental vessels. Whole mount in situ hybridization using gpr124 antisense probes was then performed on VEGF-A morphant embryos. No differences in gpr124 expression were observed between VEGF-A morpholino injected and control embryos at either 1 or 3 dpf (FIG. 32, 33). Gpr124 expression was detected in the lateral dorsal aortae and intersegmental vessels in VEGF-A morphants at 1 dpf. Expression was also detected in the pharyngeal arches later in development, similar to wild-type embryos. These findings suggest that gpr124 does not function downstream of VEGF. The reverse epistatic analysis has been performed in gpr124 knockout mice and no appreciable difference in VEGFA expression is detected in gpr124 knockout versus wild-type embryos, suggesting that at least in mice, gpr124 does not function upstream of VEGF. Taken together, these results indicate that it is unlikely that GPR124 acts within the VEGF signaling pathway. This pathway independence can lead to additive or synergistic effects of GPR124 inhibition in combination with VEGF inhibition, as relevant to anti-angiogenic therapy of cancer and ocular disorders.

### Discussion

To elucidate the mechanisms by which GPR124 regulates angiogenesis, we have analyzed GPR124 function in zebrafish. Zebrafish provide an excellent alternative to mouse for functional genomics studies and can transcend some of the limitations that exist in the mouse model system.

zgpr124 is expressed in vascular tissues during embryogenesis, first in the major trunk vessels, the dorsal aorta and axial vein, and then in the intersegmental vessels. zgpr124 is also expressed in the cranial vessels and in the aortic arch arteries. Its expression pattern closely resembles that of VE-cadherin, a known vascular endothelial specific gene. To determine GPR124 gene function in zebrafish, we used antisense morpholino oligos to knockdown protein synthesis. Initial injection of GPR124 splice morpholino resulted in embryos with reduced or completely absent pigmentation. Further analysis of GPR124 knockdown embryos revealed vascular defects, specifically angiogenic deficits within the aortic arch arteries. Morpholino injected embryos exhibited grossly abnormal aortic arch arteries, with pronounced lack of anterior extension of AA1, which supplies the cranial vasculature, and lack of lateral extension of the more posterior arch arteries. This phenotype is reminiscent of the CNS vascular phenotype observed upon GPR124 gene disruption in mice and argues for evolutionary conservation of GPR124 function as a pro-angiogenic regulatory molecule with particular tropism for the brain vasculature.

Because GPR124 is an orphan receptor, structure function analysis in mammalian systems or in vitro is extremely difficult. We used zebrafish as an in vivo system for structure function analysis of GPR124 in the absence of a known ligand. Gpr124 ectodomain RNA was injected into embryos to determine whether the ectodomain could phenocopy the morpholino knockdown, thereby demonstrating that the ectodomain is necessary and sufficient to bind ligand. Gpr124 ectodomain partially phenocopied the morpholino knockdown with angiogenic deficits in the aortic arch arteries, suggesting that GPR124 signaling is mediated through ectodomain binding of the ligand, and that the ectodomain is necessary and sufficient to bind ligand. This finding has significant implications for future experiments, providing a direction in which to study receptor signaling. GPR124 signaling through ectodomain/ligand interactions also has significant implications for soluble receptor strategies as a therapeutic approach.

Epistasis analysis revealed that GPR124 does not function downstream of the VEGF-A signaling pathway. VEGF is one of the most critical drivers of vascular formation, and although it plays a requisite role in vascular development, there are several other critical signaling pathways that are necessary for regulating angiogenesis. These pathways include Notch, Tie2/Angiopoietin, Eph/Ephrin and Hedgehog, among others.

In summary, these studies reveal an essential role for zebrafish GPR124 in patterning of the head and central nervous system vasculature. Further, zGPR124 mediates signaling through ectodomain binding of its ligand and appears to function independent of VEGF signaling.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of modulating angiogenesis in an individual, the method comprising:
administering to an individual an effective dose of an GPR124 modulating agent.

2. The method of claim 1, wherein said GPR124 modulating agent is a GPR124 antagonist, and reduces angiogenesis associated with a disorder selected from tumor growth, diabetic retinopathy, and macular degeneration.

3. The method of claim 2, wherein said agent selectively reduces activity or expression of GPR124 in pericytes.

4. The method of claim 2, wherein said agent selectively reduces activity or expression of GPR124 in the central nervous system.

5. The method of claim 1, wherein said administering is by a route selected from intravenous, in or around a solid tumor, systemic, intraarterial, intraocular, and topical.

6. The method of claim 2, wherein the GPR124 antagonist is administered in combination with a VEGF inhibitor to provide for an additive or synergistic result.

7. The method of claim 1, wherein said GPR124 modulating agent is a GPR124 agonist, and wherein said administering provides for stimulation of angiogenesis in the individual.

8. The method of claim 7, wherein said administering is effective to stimulate angiogenesis in the central nervous system.

9. The method of claim 7, wherein said administering prevents or treats ischemic stroke.
